(19) United States
(12) Patent Application Publication
(10) Pub. No.: US 2025/0235499 A1
(43) Pub. Date: Jul. 24, 2025

(54) ANTI-OVARIAN CANCER PHARMACEUTICAL COMPOSITION AND ACTION TARGET THEREOF

(71) Applicant: Zhejiang Chinese Medical University, Zhejiang (CN)

(72) Inventors: Fangfang TAO, Zhejiang (CN); Wenhong LIU, Zhejiang (CN); Ye XU, Zhejiang (CN); Mengxin ZHANG, Zhejiang (CN); Junfeng LI, Zhejiang (CN)

(73) Assignee: Zhejiang Chinese Medical University, Zhejiang (CN)

(21) Appl. No.: 19/026,546

(22) Filed: Jan. 17, 2025

(30) Foreign Application Priority Data
Jan. 18, 2024 (CN) 202410070809.5

(51) Int. Cl. A61K 36/9066; A61K 31/12; A61K 31/352; A61K 36/481; A61P 35/00

(52) U.S. Cl. CPC A61K 36/9066; A61K 31/12; A61K 31/352; A61K 36/481; A61P 35/00

(57) ABSTRACT

The present invention belongs to the pharmaceutical field, and discloses an anti-ovarian cancer pharmaceutical composition and an action target thereof. The anti-tumor pharmaceutical composition includes a Curcuma zedoaria extract and an Astragalus membranaceus extract, which synergistically enhance the efficacy by combining calycosin and bisdemethoxycurcumin to reduce the pharmaceutical dosage, minimize side effects, and save costs. An MCM2 gene, as an action target of the anti-ovarian cancer pharmaceutical composition, has been discovered. This gene has a significant impact on the proliferation, migration, and invasion abilities of ovarian cancer, providing guidance for the development and screening of a drug for preventing or treating ovarian cancer.

### CROSS-REFERENCE TO RELATED APPLICATION

This application claims the priority benefit of China application serial no. 202410070809.5, filed on Jan. 18, 2024. The entirety of each of the above-mentioned patent applications is hereby incorporated by reference herein and made a part of this specification.

### BACKGROUND

### Technical Field

The invention belongs to the field of medicines, and discloses an anti-ovarian cancer pharmaceutical composition and an action target thereof.

### Description of Related Art

The statements in this section only provide background information related to the disclosure of the present application and may not constitute the existing technology.

In gynecological tumors, the early stage symptoms of ovarian cancer are relatively insidious, and clinical screening methods are mostly unable to detect them in a timely manner. About 70% of patients are diagnosed in the advanced stage, resulting in the highest mortality rate among gynecological malignancies. The current standard treatment for ovarian cancer is surgical resection of a tumor body and platinum based chemotherapy. The pharmaceutical resistance of tumors and the serious adverse side effects after chemotherapy treatment affect the quality of life of patients, leading to a low overall survival rate.

The occurrence of tumors begins with genetic changes, and abnormal expression and activation of MCM family proteins directly damage DNA replication, leading to genomic instability and being associated with tumorigenesis and malignancy. For example, overexpression of MCM family proteins has been detected in breast cancer, hepatocellular carcinoma, lung adenocarcinoma and other tumors, which indicates that they can affect the development of tumors and have the potential to diagnose related diseases. Minichromosome maintenance protein 2 (MCM2) is an important member of the MCM protein family. When MCM2 is not expressed, it indicates that cells have exited the cell proliferation cycle, which means that the cells have differentiated and matured or entered the G0 phase. Based on this, MCM2 abnormalities can not only provide cancer warning, but also have the potential to become a target in tumor treatment. MCM2 is also a biomarker of proliferation, migration, invasion, and metastasis in tumor progression, and is positively correlated with TNM staging and lymph node metastasis in many cancers. Studies on cholangiocarcinoma have shown that overexpression of MCM2 promotes the progression of cholangiocarcinoma (CCA) by inhibiting a p53 signaling pathway. The increase in MCM2 level accelerates the proliferation, migration, and invasion of tumor cells, and inhibits cell apoptosis. In “Thiabendazole Inhibits Glioblastoma Cell Proliferation and Invasion Targeting Mini-chromosome Maintenance Protein 2”, Y-T Hu et al. found that upregulation of MCM2 promotes proliferation, migration, and invasion of medulloblastoma (MB) cells. The existing technology “inhibiting the expression of microchromosome support protein 2 (MCM2) can enhance the sensitivity of ovarian cancer to carboplatin” discloses that the sensitivity of an ovarian cancer cell to carboplatin may be related to the P53 mediated apoptosis mechanism. Downregulating the expression of MCM2 can improve the sensitivity of the ovarian cancer cell to chemotherapy, providing a new method for treating ovarian cancer.

In recent years, with the in-depth research on traditional Chinese medicine, it has played an important role in the treatment of tumors and has shown good therapeutic effects on tumor diseases. At the same time, it has also shown good improvement in the adverse reactions caused by chemotherapy, improving the quality of life of patients. Due to the wide variety and diverse effects of active ingredients in traditional Chinese medicine, exploring the mechanism of action of extracting and combining active ingredients in traditional Chinese medicine on tumor treatment can provide theoretical basis for the development of low side effect and efficient tumor treatment pharmaceuticals, and is also of great significance for the development and utilization of anti-tumor traditional Chinese medicine.

### SUMMARY

The purpose of the present invention is to provide an anti-ovarian cancer pharmaceutical composition and an action target thereof in response to the shortcomings of current anti-tumor drugs and methods, providing effective traditional Chinese medicine ingredients for the fight against ovarian cancer and guiding the development of anti-ovarian cancer drugs.

The technical solution of the present invention is as follows:

An anti-ovarian cancer pharmaceutical composition comprising a Curcuma zedoaria extract.

According to a preferred embodiment, the anti-ovarian cancer pharmaceutical composition further comprises an Astragalus membranaceus extract.

According to a preferred embodiment, the Astragalus membranaceus extract comprises calycosin, and the Curcuma zedoaria extract comprises bisdemethoxycurcumin.

According to a preferred embodiment, a ratio of the calycosin to the bisdemethoxycurcumin is 16:1.

According to a preferred embodiment, the anti-ovarian cancer pharmaceutical composition targets MCM2.

Another aspect of the present invention also provides use of an MCM2 gene as an action target of the anti-ovarian cancer pharmaceutical composition in the development and screening of a drug for preventing or treating ovarian cancer, wherein the pharmaceutical composition targets the MCM2 gene to affect cell cycle regulation and inhibit an ovarian cancer cell by an EMT pathway.

According to a preferred embodiment, the pharmaceutical composition affects the levels of CDK4, Cyclin D1, and P21, causing the formation of Cyclin D1-CDK4/6 complex and blocking the cell cycle.

According to a preferred embodiment, the pharmaceutical composition affects the levels of E-cadherin, N-cadherin, and Vimentin to regulate the EMT pathway, reducing the proliferation, migration, and invasion abilities of an ovarian cancer cell.

The beneficial effects of the present invention compared to the existing technology are:
1. an anti-ovarian cancer pharmaceutical composition, the existing technology provides use of a long-acting curcumin derivative in the preparation of an anti-tumor drug, and discloses that curcumin, demethoxycurcumin or bisdemethoxycurcumin can inhibit tumors such as ovarian cancer and cervical cancer when acting alone; in the use of the pharmaceutical composition, the synergistic effect of different pharmaceutical compositions can significantly enhance the effectiveness of the pharmaceutical compositions while reducing the dosage and saving costs; however, the inventors of the present application have found that the combination of calycosin and bisdemethoxycurcumin has a synergistic effect, which can synergistically combat ovarian cancer and produce better inhibitory effects than using bisdemethoxycurcumin alone, and the combination of the two can reduce drug dosage, lower drug side effects, and save costs in combating ovarian cancer; and
2. use of an MCM2 gene as an action target of the anti-ovarian cancer pharmaceutical composition in the development and screening of a drug for preventing or treating ovarian cancer. Through research and analysis, it is found that the MCM2 gene inhibits the proliferation, migration, cloning, and invasion of an ovarian cancer cell by affecting the levels of CDK4, Cyclin D1, and P21, causing the formation of a Cyclin D1-CDK4/6 complex, blocking the cell cycle, and regulating the EMT pathway by affecting the levels of E-cadherin, N-cadherin, and Vimentin; compared with the existing technology, a new mechanism of action for the MCM2 gene is provided; and it is confirmed that the MCM2 gene also serves as a drug target for the anti-ovarian cancer effects of genistein and bisdemethoxycurcumin; and a guidance for screening the anti-ovarian cancer pharmaceutical composition is provided.

### BRIEF DESCRIPTION OF THE DRAWINGS

FIGS. 1A-1D show the effects of different concentrations of CA and BDMC on the proliferation of SKOV3 and Hey ovarian cancer cells in Example 1;

FIGS. 2A and 2B show the inhibition rate and combination coefficient of CA and BDMC combined administration in Example 1;

FIGS. 3A-3C are box plots of the normalization of sample data in each data chip in Example 2;

FIG. 4 shows a Venn map of differentially expressed genes among three data chips in Example 2;

FIG. 5 shows a bubble plot of KEGG gene pathway enrichment results in Example 2;

FIG. 6 shows the key functional modules of the protein interaction network in Example 2;

FIG. 7 shows the survival analysis of FAM83D and MCM2 genes in Example 2;

FIG. 8 shows the expression of MCM2 protein and mRNA in SKOV3 and Hey cells after MCM2 knockdown in Example 3 (x±s, n=3). Note: ** P<0.01;

FIG. 9 shows the expression of MCM2 protein and mRNA in SKOV3 and Hey cells after MCM2 overexpression in Example 3 (x±s, n=3). Note: ** P<0.01;

FIGS. 10A-10D show the changes in proliferation ability of SKOV3 and Hey cells after MCM2 knockdown and overexpression in Example 3;

FIGS. 11A-11D show the changes in clone formation of SKOV3 and Hey cells after MCM2 knockdown and overexpression in Example 3 (x±s, n=3);

FIGS. 12A-12D show the migration changes of SKOV3 and Hey cells after MCM2 knockdown and overexpression in Example 3 (x±s, n=3);

FIGS. 13A-13D show the invasion changes of SKOV3 and Hey cells after MCM2 knockdown and overexpression in Example 3 (x±s, n=3);

FIGS. 14A-14D show the changes in tumor volume and final tumor inhibition rate (x±s, n=8) of tumor bearing nude mice after intervention with DDP, CA, BDMC, and combined administration in Example 1;

FIGS. 15A and 15B show the docking diagram of CA and BDMC with MCM2 protein molecules in Example 4;

FIGS. 16A and 16B show MCM2 protein expression in SKOV3 and Hey cells intervened by CA and BDMC in Example 4 (x±s, n=3);

FIGS. 17A and 17B show the clone formation of MCM2 knockdown SKOV3 and Hey cells intervened by CA, BDMC, and combined administration in Example 4 (x±s, n=3);

FIGS. 18A and 18B show the colony formation of SKOV3 and Hey cells with MCM2 overexpression intervened by CA, BDMC, and combined administration in Example 4 (x±s, n=3);

FIGS. 19A-19D show the changes in cell migration and invasion of MCM2 knockdown SKOV3 and Hey cells after intervention with CA, BDMC, and combined administration in Example 4 (x±s, n=3);

FIGS. 20A-20D show the changes in cell migration and invasion of SKOV3 and Hey cells with MCM2 overexpression after intervention with CA, BDMC, and combined administration in Example 4 (x±s, n=3);

FIGS. 21A-21D show the changes in cell cycle of SKOV3 and Hey cells after MCM2 knockdown and overexpression in Example 5 (x±s, n=3);

FIGS. 22A-22D show the expression of cell cycle related proteins in SKOV3 and Hey cells after MCM2 knockdown and overexpression in Example 5 (x±s, n=3);

FIGS. 23A-23D show the expression of EMT related proteins in SKOV3 and Hey cells after MCM2 knockdown and overexpression in Example 5 (x±s, n=3);

FIGS. 24A and 24B show the percentage of G0/G1 phase cells of MCM2 knockdown SKOV3 and Hey cells intervened by CA, BDMC, and combined administration in Example 5 (x±s, n=3);

FIGS. 25A and 25B show the percentage of G0/G1 phase cells of SKOV3 and Hey cells with MCM2 overexpression after intervention with CA, BDMC, and combined administration in Example 5 (x±s, n=3);

FIGS. 26A-26D show the changes in cell cycle and EMT related proteins of MCM2 knockdown SKOV3 and Hey cells after intervention with CA, BDMC, and combined administration in Example 5 (x±s, n=3);

FIGS. 27A-27D show the changes in cell cycle and EMT related proteins of SKOV3 and Hey cells with MCM2 overexpression after intervention with CA, BDMC, and combined administration in Example 5 (x±s, n=3);

FIG. 28 shows the immunohistochemistry of cyclin in a nude mouse tumor body cell in Example 5 (400×);

FIG. 29 shows the expression of a tumor body cyclin in tumor bearing nude mice in Example 5;

FIG. 30 shows the expression of a tumor body EMT protein in tumor bearing nude mice in Example 5.

### DESCRIPTION OF THE EMBODIMENTS

Below is a further detailed description of the features and performance of the present invention in conjunction with the embodiments. Calycosin is abbreviated as CA; and bisdemethoxycurcumin is abbreviated as BDMC. A human SKOV3 ovarian cancer cell, a human Hey ovarian cancer cell and a human 293T cell used in the experiment are all purchased from the cell bank of the Chinese Academy of Sciences. Unless otherwise specified, the experimental materials and instruments used in the present application are commercially available or conventional reagents in this field. Unless otherwise specified, the experimental methods used in the present application are conventional methods in this field.

### Example 1 Synergistic Effect of CA and BDMC on an Ovarian Cancer Cell

### 1. CCK-8 Detection of Proliferation of an Ovarian Cancer Cell

SKOV3 and Hey ovarian cancer cells in logarithmic growth phase were digested with 0.1% trypsin to prepare a cell suspension, and the cell concentration was quantified at 10⁴ cells/mL. 200 μL of the cell suspension each well was inoculated into a 96 well plate, and 200 μL of PBS was added to each well of the outer circle to eliminate edge effects. The 96 well plate was placed in a 37° C. incubator containing 5% CO₂ for 24 hours each. After the cells adhered to the wall, the culture medium was discarded, and the cells were washed three times with PBS buffer. New culture medium containing different concentrations of CA (0, 50, 100, 200, 400, and 800 μmol/L) and BDMC (0, 3.13, 6.25, 12.5, 25, and 500 μmol/L) were added, with 6 parallel wells in each group. After adding a pharmaceutical, cultivation was further conducted, the 96 well plate was taken out at 24 hours and 48 hours, the original culture medium was discarded, the cells were washed three times with PBS, 200 μL of prepared serum-free culture medium containing 10% CCK-8 was added, and incubated at 37° C. for 1 hour. A well with drug concentration of 0 μmol/L was used as a control well and a well without cells and only added with a CCK-8 solution as a blank well. An absorbance at a wavelength of 450 nm was measured using a microplate reader, and an IC50 value was calculated using GraphPad Prism 8 software. Cell survival rate (%)=[(control well−experimental well)/(control well−blank well)]×100%.

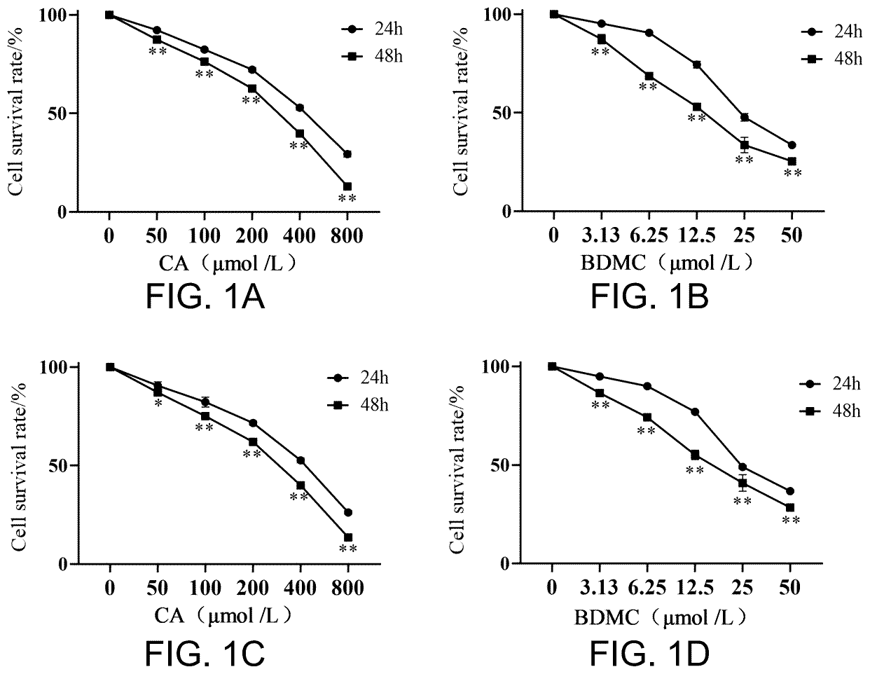

The detection results were shown in FIGS. 1A-1D, wherein FIGS. 1A and 1B illustrated the effects of CA and BDMC on the proliferation of the SKOV3 ovarian cancer cell; and FIGS. 1C and 1D show the effects of CA and BDMC on the proliferation of the Hey ovarian cancer cell.* P<0.05, ** P<0.01. From FIGS. 1A-1D, it could be seen that the growth of SKOV3 and Hey ovarian cancer cells treated with CA at 800, 400, 200, 100, and 50 μmol/L and BDMC at 50, 25, 12.5, 6.25, and 3.125 μmol/L, respectively, was inhibited compared to the control group. The results showed that both CA and BDMC had proliferative inhibitory effects on SKOV3 and Hey ovarian cancer cells, and their proliferative inhibitory effects increased with increasing drug concentration during the same time period, showing a concentration dependence. At the same concentration, the proliferation inhibition rate of the 48-hour intervention group was significantly higher than that of the 24-hour intervention group. The proliferation inhibition effects of CA and BDMC on SKOV3 and Hey ovarian cancer cells were time-dependent.

### 2. Evaluation of the Synergistic Effect of CA and BDMC on an Ovarian Cancer Cell

A pharmaceutical synergistic effect experiment was conducted based on the IC50 values of CA and BDMC on the proliferation inhibition rates of SKOV3 and Hey ovarian cancer cells, respectively.

The SKOV3 and Hey ovarian cancer cells in logarithmic growth phase were digested with 0.1% trypsin to prepare a cell suspension, and the cell concentration was quantified at 10⁴ cells/mL. 200 μL of the cell suspension each well was inoculated into a 96 well plate, with 6 parallel wells in each group. The 96 well plate was placed in a 37° C. incubator containing 5% CO₂ for 24 hours each. After the cells adhered to the wall, the culture medium was discarded, and the cells were washed three times with PBS buffer. 200 μL of a mixed drug solution containing CA (0, 50, 100, 200, 400, and 800 μmol/L) and BDMC (0, 3.13, 6.25, 12.5, 25, and 50 μmol/L) were added respectively, and set up zero adjustment and blank wells, with 6 parallel wells in each group. The 96 well plate was placed in an incubator, and cultivation was further conducted for 24 hours. After cultivation, cultivation was further conducted, the original culture medium was discarded, the cells were washed three times with PBS, 200 μL of prepared serum-free culture medium containing 10% CCK-8 was added, and incubated at 37° C. for 1 hour. A well with drug concentration of 0 μmol/L was used as a control well and a well without the cells and only added with a CCK-8 solution as a blank well. An absorbance at a wavelength of 450 nm was measured using a microplate reader, and an inhibition rate was calculated. A combination index (CI) of two drugs were calculated using Compusyn software and Chou-Talalay data processing model. When CI=1, the two drugs had an additive effect; when CI>1, the two drugs had an antagonistic effect; and when CI<1, the two drugs had a synergistic effect.

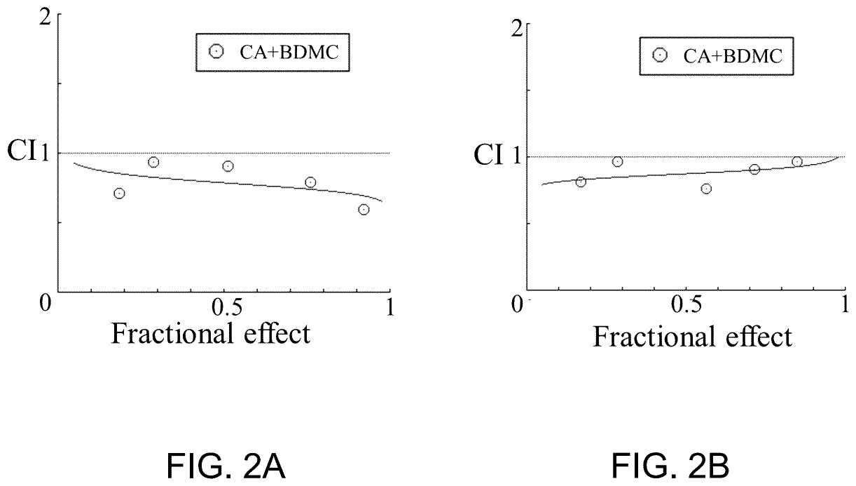

The test results were shown in FIGS. 2A and 2B and Tables 1 and 2. FIG. 2A showed the cell inhibition rate and combination coefficient of the combined action of CA and BDMC on SKOV3 ovarian cancer cells; and FIG. 2B showed the cell inhibition rate and combination coefficient of the combined action of CA and BDMC on Hey ovarian cancer cells.

TABLE 1
CA and BDMC combined administration effect on SKOV3
cell inhibition rate and combination coefficient

| Combination drug dosage | Inhibition rate | Combination coefficient CI |
|---|---|---|
| CA 800 μmol/L + BDMC 50 μmol/L | 92.26% | 0.59683 |
| CA 400 μmol/L + BDMC 25 μmol/L | 76.21% | 0.7949 |
| CA 200 μmol/L + BDMC 12.5 μmol/L | 51.37% | 0.91164 |
| CA 100 μmol/L + BDMC 6.25 μmol/L | 28.81% | 0.9358 |
| CA 50 μmol/L + BDMC 3.125 μmol/L | 18.71% | 0.71509 |

TABLE 2
CA and BDMC combined administration effect on Hey
cell inhibition rate and combination coefficient

| Combination drug dosage | Inhibition rate | Combination coefficient CI |
|---|---|---|
| CA 800 μmol/L + BDMC 50 μmol/L | 84.98% | 0.96576 |
| CA 400 μmol/L + BDMC 25 μmol/L | 71.62% | 0.90859 |
| CA 200 μmol/L + BDMC 12.5 μmol/L | 56.51% | 0.76445 |
| CA 100 μmol/L + BDMC 6.25 μmol/L | 28.49% | 0.96644 |
| CA 50 μmol/L + BDMC 3.125 μmol/L | 17.04% | 0.81297 |

When CA: BDMC at a ratio of 16:1 intervened in SKOV3 and Hey ovarian cancer cells, a combination index (CI) value of the two pharmaceuticals were all below 1 at different dosages, indicating a synergistic effect of the two drugs.

### 3. Animal Experiment Detected the Synergistic Effect of CA and BDMC on Ovarian Cancer Cells

### (1) Construction of a Tumor Bearing Nude Mouse Model

Newly housed BALB/c nude mice were placed in an SPF grade feeding room laminar flow rack for adaptive feeding for one week, and the experimental animal status was adjusted. A Hey ovarian cancer cell was cultivated to the logarithmic growth stage, and the original culture medium was discarded. The Hey ovarian cancer cell was washed with PBS buffer, added with 0.1% trypsin digestion for digestion, added with the corresponding culture medium to terminate digestion, centrifuged at 1000 rpm for 3 minutes, and added with PBS buffer to prepare a cell suspension. The cell was counted using a hemocytometer, and the cell concentration was quantified to 5×10⁷ cells/mL. After grasping and fixing the nude mouse with the left hand, and the skin at a needle inserting site on the right forelimb axilla of the nude mouse was disinfected with a 75% alcohol cotton ball. A disposable 1 mL syringe was used to inject approximately 100 μL of cell suspension subcutaneously into the posterior axilla of the right forelimb in an oblique stabbing inserting needle mode, so that each nude mouse was injected with approximately 5×10⁶ cells. After vaccination, the needle inserting site was gently pressed with a cotton ball to prevent the cell suspension from leaking from a needle hole.

### (2) Administration Treatment for Tumor Bearing Nude Mice

The growth status of tumors at the vaccination site of nude mice was observed daily. After about one week, if the tumors at the vaccination site were compacted and appeared light red in color, it could be considered as successful tumor growth. At this time, the nude mice were randomly divided into 5 groups, a Control group intraperitoneally injected with physiological saline for 1 day each time; a DDP group intraperitoneally injected with cisplatin, 3 mg/kg, 3 d/time; a CA group intraperitoneally injected with CA, 80 mg/kg, 1 d/time; a BDMC group intraperitoneally injected with BDMC, 5 mg/kg, 1 d/time; a CA+BDMC group intraperitoneally injected with CA 80 mg/kg+BDMC 5 mg/kg, 1 d/time, 8 animals per group.

A tumor length (L) and a tumor length (W) were measured with a vernier caliper every 3 days to calculate a tumor volume. After 15 days of administration, nude mice were euthanized by dislocation of cervical vertebra, and tumor tissue was dissected. The tumor weight was weighed to calculate the tumor inhibition rate. The tumor tissue was stored in two batches at −80° C. and fixed with 4% paraformaldehyde.

$$Tumor volume V = \frac{Long diameter L \times Short diameter {W}^{2}}{2}$$

$$Tumor inhibition rate ( \% ) = \frac{Control group tumor weight - Administration group tumor weight}{Control group tumor weight} \times 100 \%$$

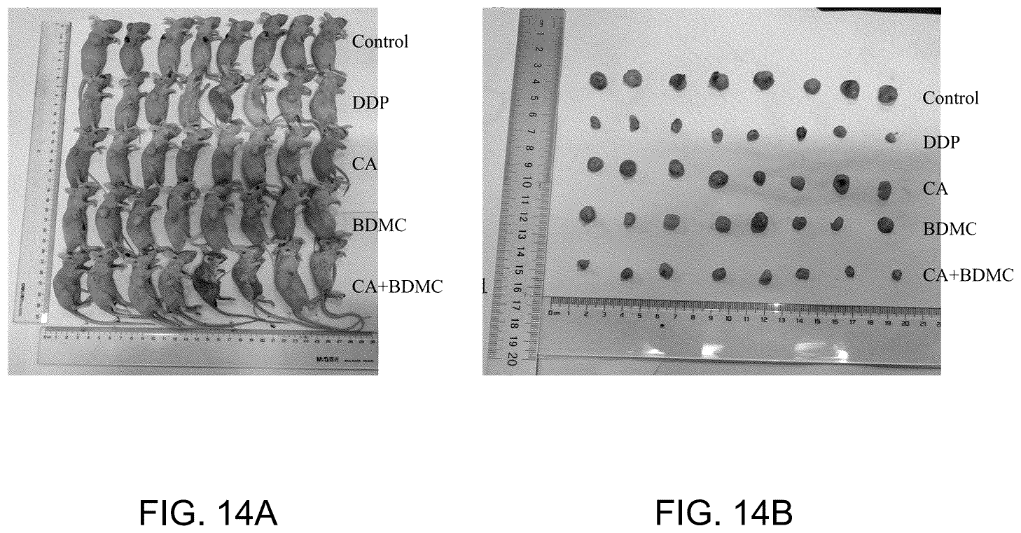

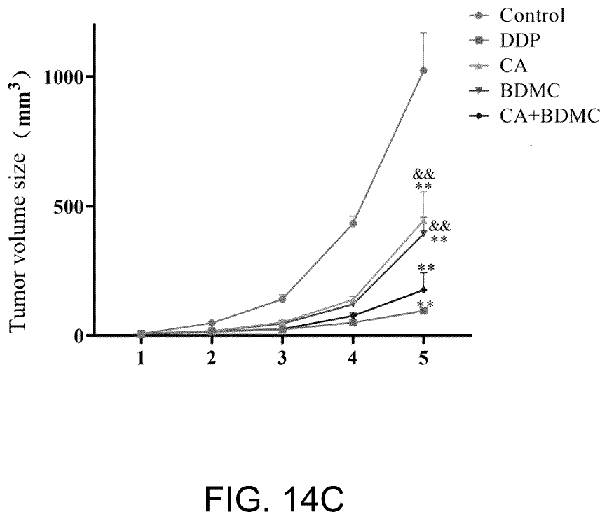

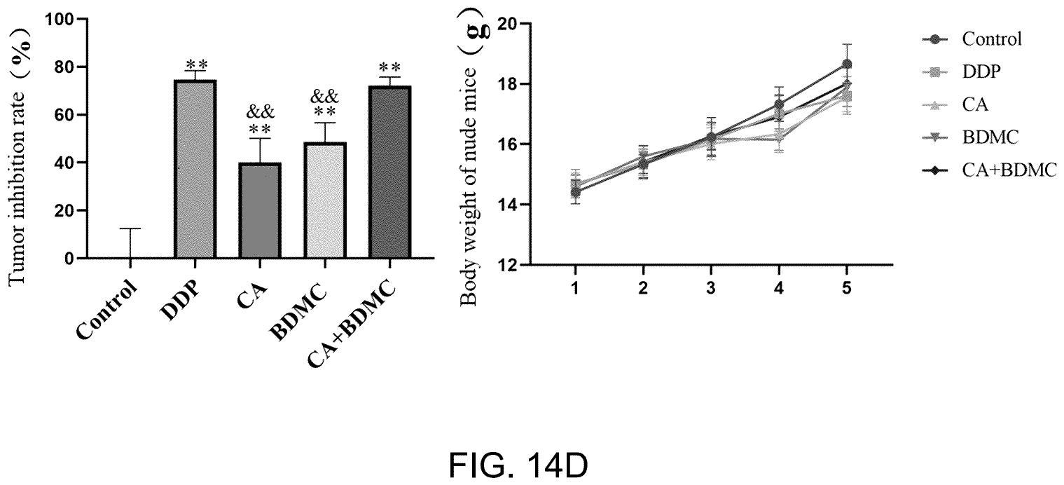

After intervention with DDP, CA, BDMC, and combined administration, the tumor volume changes and final tumor inhibition rate of the tumor bearing nude mice were shown in FIGS. 14A-14D, wherein A: nude mouse images of each group; B: tumor images of nude mice in each group; C: a tumor volume size measured by each group each time; D: a final tumor inhibition rate of each group; E: a body weight of nude mice measured in each group. Compared with the Control group, ** P<0.01; and compared with the CA+BDMC group, &&P<0.01.

As shown in FIGS. 14A-14D, the tumor volume size (FIG. 14C) was significantly reduced in the DDP group, CA group, BDMC group, and CA+BDMC group compared to the Control group (P<0.01); and the CA+BDMC group showed a significant decrease compared to the CA group and BDMC group (P<0.01). The tumor inhibition rate (FIG. 14D) was significantly increased in the DDP group, CA group, BDMC group, and CA+BDMC group compared to the Control group (P<0.01); and the CA+BDMC group showed a significant increase compared to the CA group and BDMC group (P<0.01). This experiment showed that CA, BDMC, and combined administration could inhibit tumor body proliferation in the tumor bearing nude mice, and the combined administration was more effective than single drug administration. There was no significant difference in body weight between the groups.

### Example 2 Screening of MCM2 Gene, a Key Gene that Plays a Role in the Proliferation, Migration, Invasion, and Cell Cycle of an Ovarian Cancer Cell

### 1. GEO Database Download and Data Preprocessing

After entering the GEO database from website https://www.ncbi.nlm.nih.gov/geo/, screening was performed according to the following criteria: (1) a sample was sourced from ovarian cancer patient tissue; (2) the sample needed to contain both normal ovarian tissue and ovarian cancer sample; (3) the more samples, the better. A required chip was selected for download based on the information contained in the chip. Three data chips were filtered and downloaded, the background of each sample was processed through signal value, filled in missing data, performed normalization processing of data, and subsequent differential gene comparison.

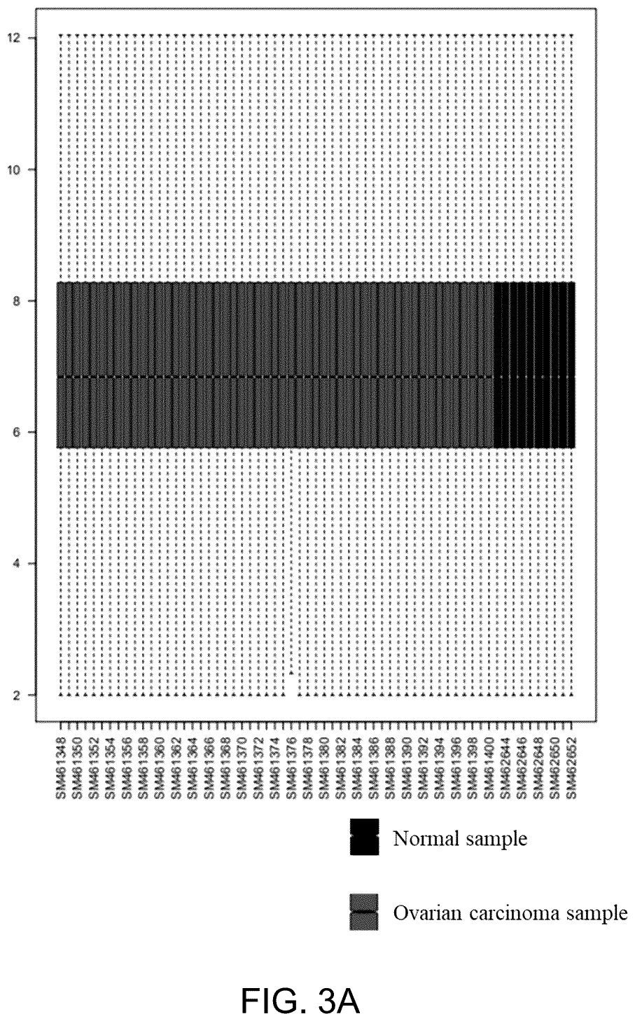

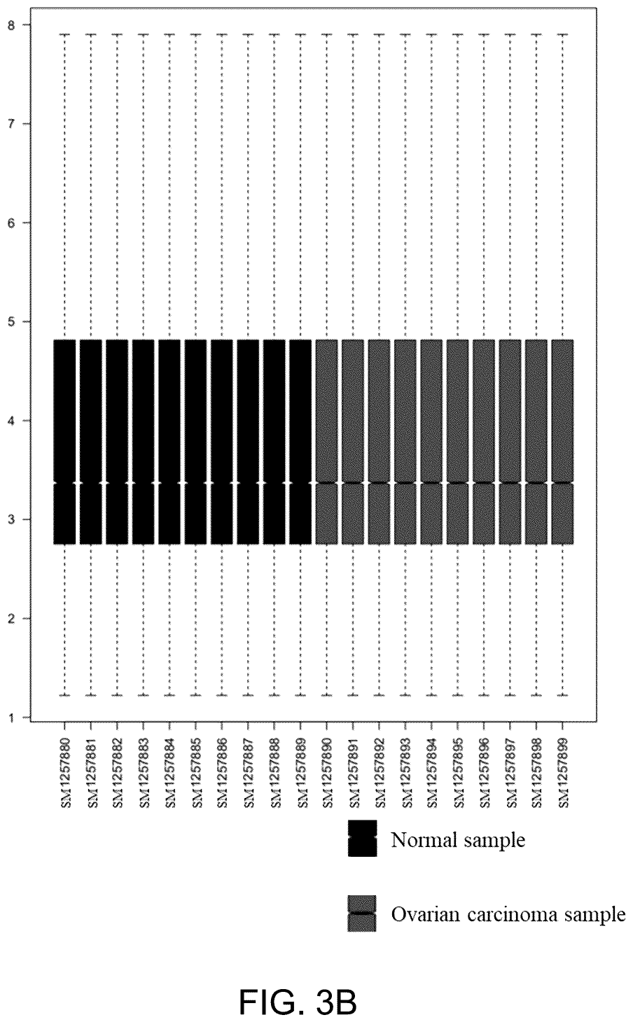

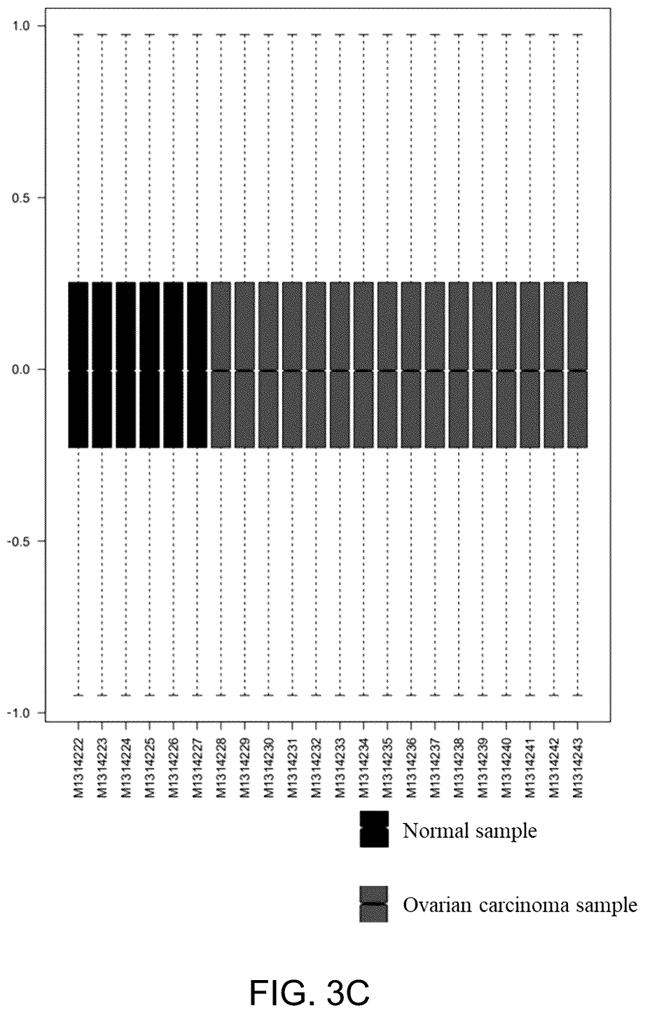

As shown in FIGS. 3A-3C. The median of each sample was basically on the same horizontal line, indicating a good degree of normalization between the samples, which could be used for subsequent differential gene comparison.

### 2. Differential Gene Enrichment Analysis

Using a KEGG database, the obtained differential gene was imported and screened for the biological characteristics and signaling pathways of differential genes between ovarian cancer and normal ovarian tissues.

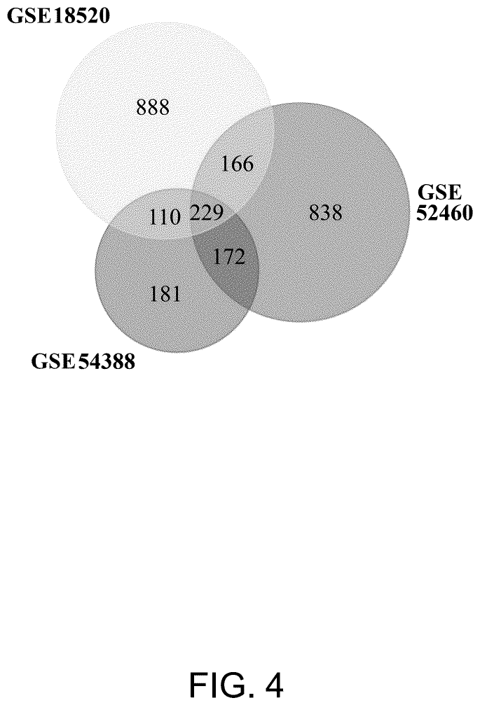

As shown in FIG. 4, there were a total of 229 genes at the intersection of the three data chip sets, indicating that there were 229 differential genes shared by the three data chips.

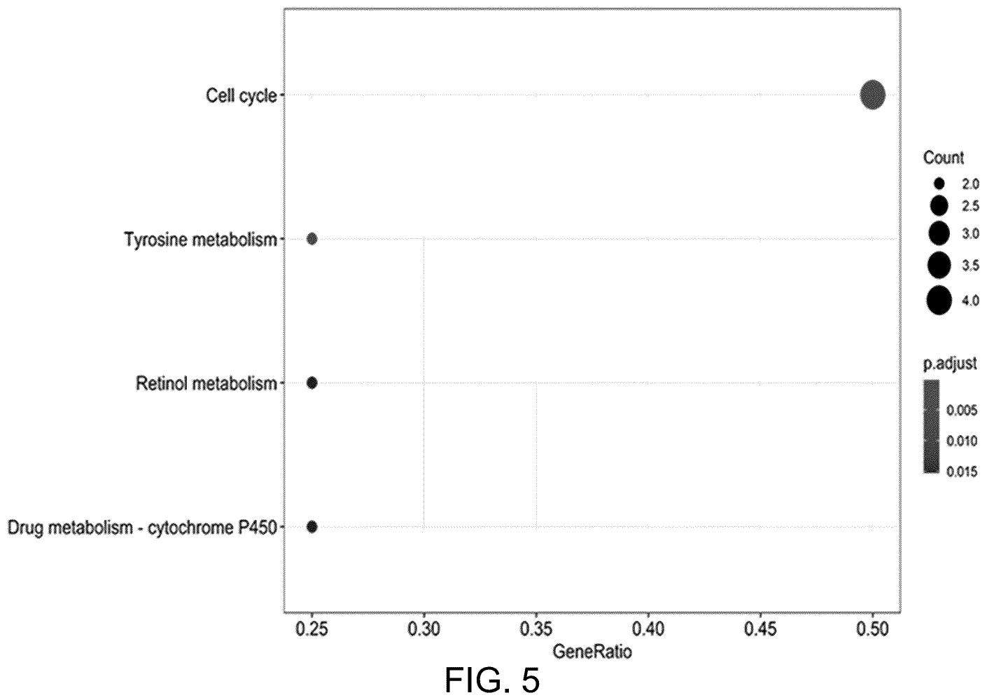

The functions of 229 differentially expressed genes were further explored, and the differential genes co-expressed by the three data chips were imported into a KEGG database for signal pathway enrichment analysis. The results were shown in FIG. 5 and Table 3. The size of the bubbles in FIG. 5 represented the number of genes involved in the pathway, and the colors from blue to red indicated the P-values from high to low. The differentially expressed genes obtained mainly exerted their biological functions through the following four signaling pathways: Cell cycle, Tyrosine metabolism, Retinol metabolism, and Drug metabolism cytochrome P450. Cell cycle had the highest correlation.

TABLE 3
KEGG pathway analysis results

| | Number of | | |
|---|---|---|---|
| Pathway | genes | P value | Containing genes |
| Cell cycle | 4 | 3.4848e−6 | TTK\grave{ }MCM2\grave{ } |
| | | | CCNB1\grave{ }BUB1B |
| Tyrosine metabolism | 2 | 0.0005 | MAOB\grave{ }ADH1B |
| Retinol metabolism | 2 | 0.0018 | ALDH1A3\grave{ }ADH1B |
| Drug metabolism - | 2 | 0.0021 | MAOB\grave{ }ADH1B |
| cytochrome P450 | | | |

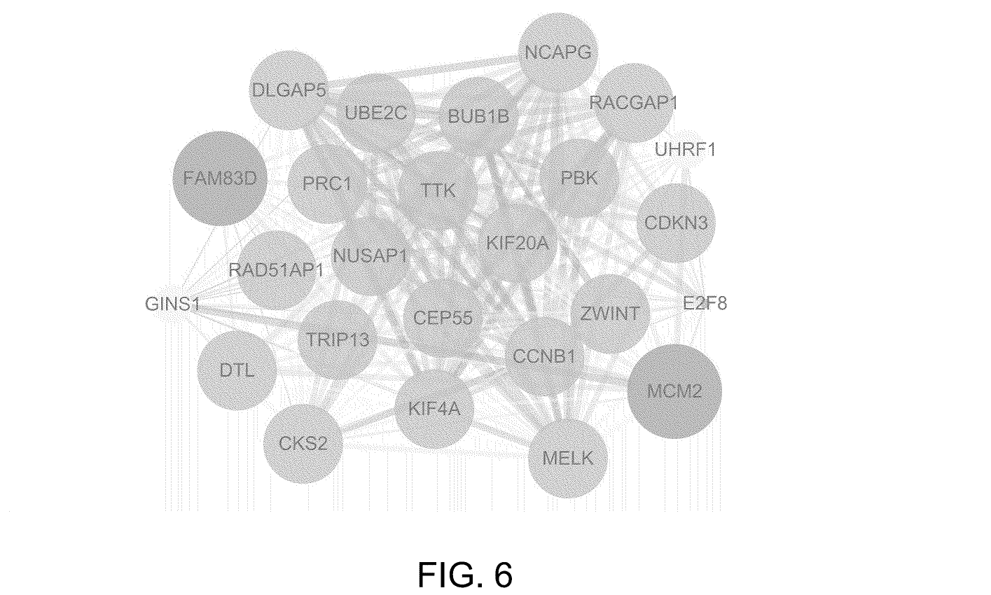

Using a STRING database, 229 differential genes were imported, and “Homo sapiens” was selected from the species category for analysis of the interaction network of the differential genes. By analyzing the protein interaction network, key node proteins in the network could be screened. A Cytoscape software was used for visualization and MCODE plugin in the software, with “Degree Cutoff” set to 2 and “Node Score Cutoff” set to 0.2 for screening key modules in the protein interaction network. The protein interaction network data was obtained. The protein interaction data constructed from the STRING database was visualized using the Cytoscape software, and key functional module analysis was performed using the MCODE plugin. A key module containing 25 node proteins and 286 protein pairs was obtained, as shown in FIG. 6. The larger the bubble volume in FIG. 6, the darker the color, indicating more interacted proteins. The proteins expressed by these differential genes might be key sites that were affected or exerted activation and inhibition effects during the process of normal ovarian cancer tissue carcinogenesis and ovarian cancer progression.

### 3. Survival Analysis of Key Genes

The genes in the key modules obtained were input into a KM Plotter database, “ovarian cancer” in the disease was selected, the gene names in the key modules was input in “Use multiple genes”, the survival option of “OS” as the overall survival rate was selected in “survival” for performing survival analysis, and “Draw Kaplan Meier plot” was selected to output the survival curve.

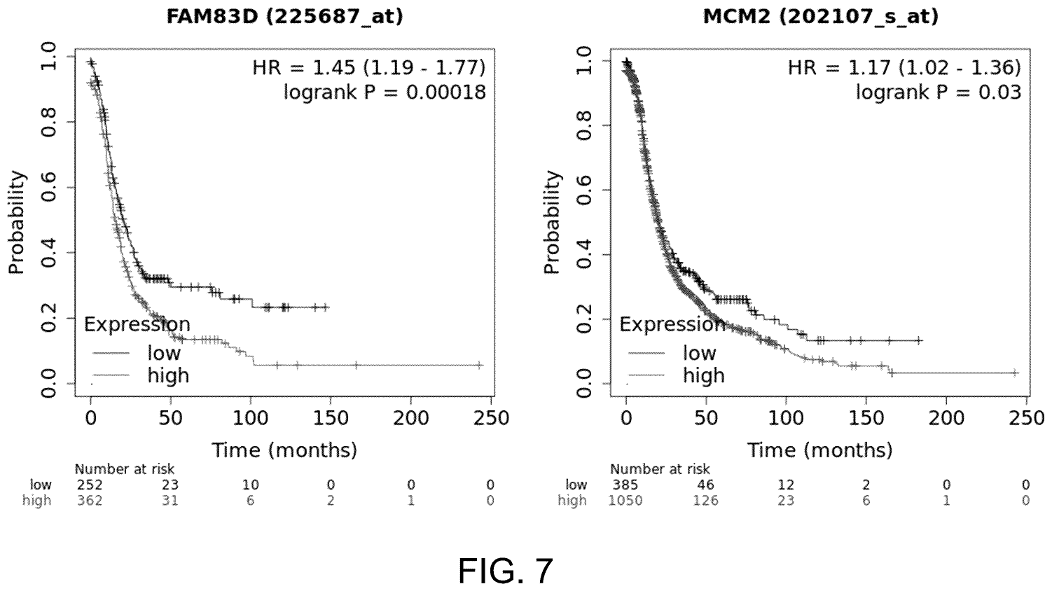

The survival analysis was performed using a Kaplan Meier Plotter database on the genes of the key functional modules selected by the MCODE plugin in the Cytoscape software to predict their association with ovarian cancer prognosis. The K-M curve was shown in FIG. 7. The results showed that the key genes FAM83D and MCM2 in the module were significantly correlated with survival (P<0.05).

### Example 3 Effects of MCM2 Gene on Proliferation, Migration, Invasion, and Cell Cycle of an Ovarian Cancer Cell

### 1. The Effect of MCM2 Gene on the Proliferation of an Ovarian Cancer Cell

### (1) MCM2 Knockdown and Overexpression in SKOV3 and Hey Ovarian Cancer Cells Infected by Lentivirus

SKOV3 and Hey ovarian cancer cells were passaged and cultured in a 6 cm dish. When they grew to 50%-60% and showed good growth, lentivirus infection was performed. SKOV3 and Hey ovarian cancer cells were divided into five groups: MCM2 knockdown group (sh-MCM2), MCM2 knockdown virus negative control group (sh-NC), MCM2 overexpression group (OE-MCM2), MCM2 overexpression virus negative control group (OE-NC), and blank control group (Control). MCM2 knockdown and a corresponding negative virus thereof, as well as MCM2 overexpression and a corresponding negative virus thereof were taken out from a −80° C. freezer and placed on ice for natural melting. SKOV3 and Hey ovarian cancer cell culture dishes were taken out from each group, an appropriate volume of their corresponding lentivirus solution was added to each dish, further placed to culture in an incubator for 24 hours to infect the cells with lentivirus. After the virus infection time was over, the original culture medium was aspirated, and 3 mL PBS buffer was added. A culture bottle was gently shaken to completely cover and wash the cells 3-4 times, PBS buffer was poured out, the operation was repeated once, and a corresponding culture medium containing 10% FBS was added to continue to culture for 24 hours. After the cultivation time was over, the original culture medium was aspirated and replaced with a corresponding culture medium containing 2 μg/mL puromycin in 10% FBS. After the cells in the control group were completely dead, the culture medium of other groups was discarded. The cells were washed twice with PBS and added with a culture medium containing 10% FBS for further cultivation. The fluorescence expression of the cells was observed under an inverted microscope, and the expression of MCM2 protein and mRNA was detected by Western Blot and RT-qPCR experiments. The infected cells were passaged, amplified, and frozen for future experiments.

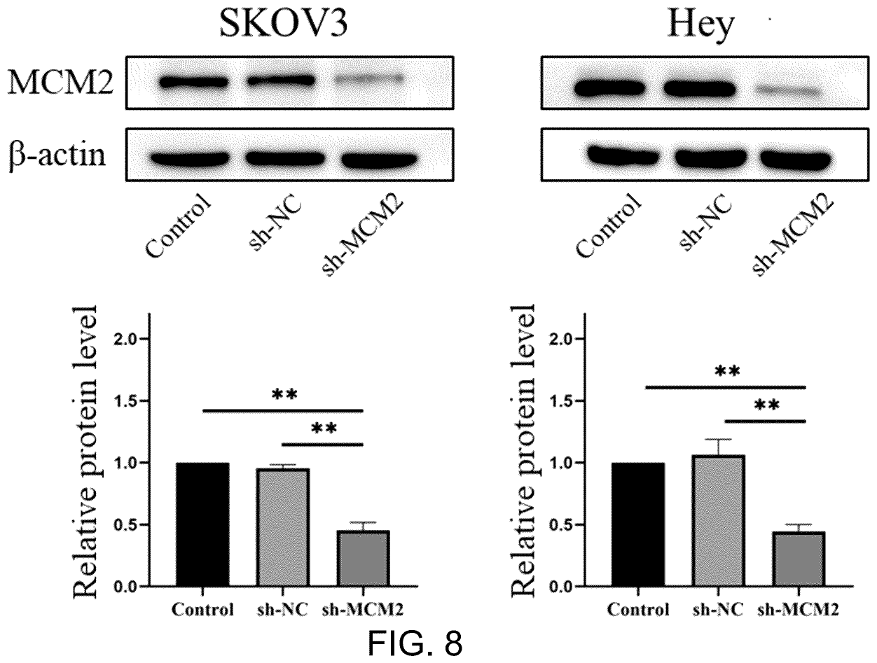

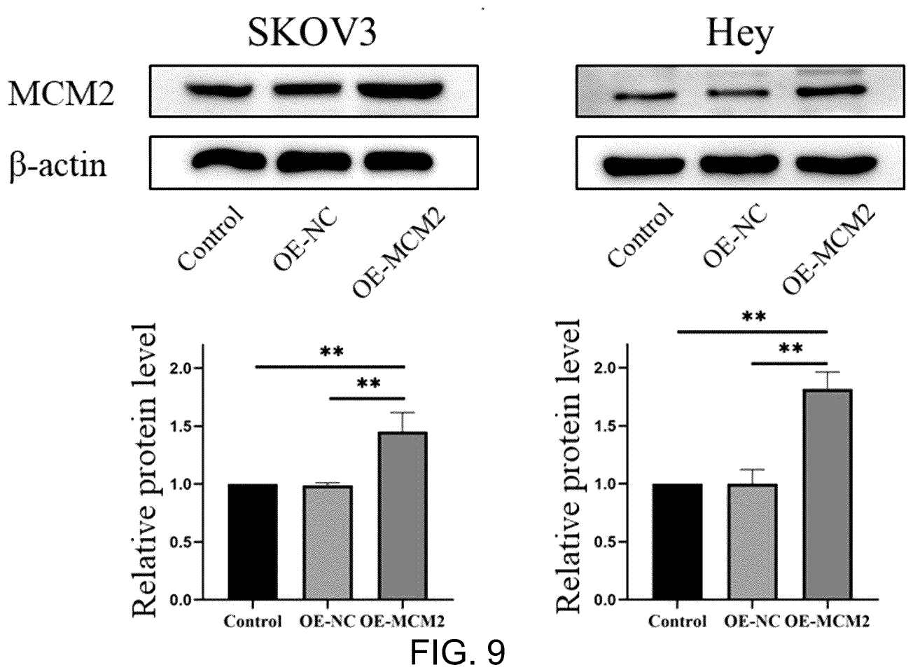

The results of MCM2 knockdown were shown in FIG. 8. After SKOV3 and Hey ovarian cancer cells were infected with lentivirus and knocked down MCM2, the MCM2 protein and mRNA levels in the sh-MCM2 group were significantly decreased compared to the Control group and sh-NC group (P<0.01); and there was no significant change in the levels of the MCM2 protein and mRNA in the Control group and sh-NC group cells. The overexpression results of MCM2 were shown in FIG. 9. SKOV3 and Hey ovarian cancer cells were infected with lentivirus and overexpressed with MCM2. The MCM2 protein and mRNA levels of the OE-MCM2 group cell were significantly increased compared with the Control group and OE-NC group (P<0.01); and there was no significant change in the levels of the MCM2 protein and mRNA levels in the Control group and OE-NC group cells.

### (2) CCK-8 Experiment Detected Cell Proliferation

SKOV3 and Hey ovarian cancer cells in logarithmic growth phase were knocked down with MCM2 (Control group, sh-NC group, sh-MCM2 group) and overexpressed with MCM2 (Control group, OE-NC group, OE-MCM2 group). The original culture medium was discarded, washed with PBS buffer, and digested with 0.1% trypsin. A corresponding culture medium was added to prepare a cell suspension. Cells were counted using a hemocytometer and a cell concentration was quantified at 10⁴ cells/mL. 200 μL of each well was inoculated into a 96 well plate, with 6 parallel wells in each group, and incubated in an incubator containing 5% CO₂ at 37° C. for 0, 24, 48, and 72 hours, respectively. After a corresponding cultivation time was over, a serum-free culture medium containing 10% CCK-8 was prepared in advance, the corresponding 96 well plate was taken out, the original culture medium was carefully aspirated, and the cells were gently washed three times with a PBS buffer. 200 μL of the corresponding prepared 10% CCK-8 mixed solution was added to each well, incubated at 37° C. for 1 hour in a constant temperature incubator, and an absorbance (OD value) at 450 nm wavelength was measured using a microplate reader to detect cell proliferation at the above-mentioned time.

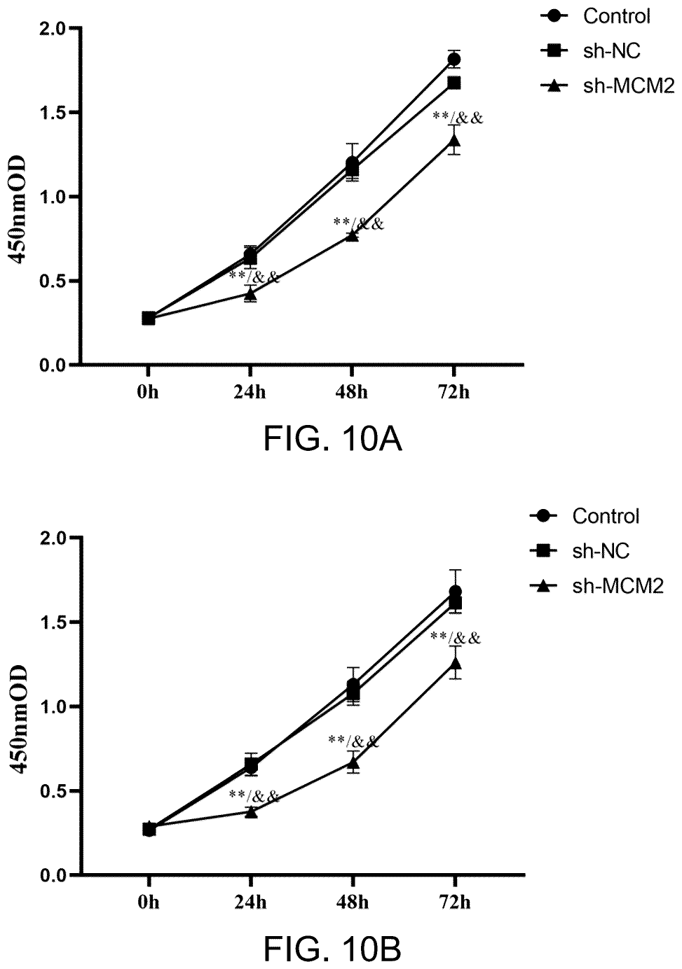

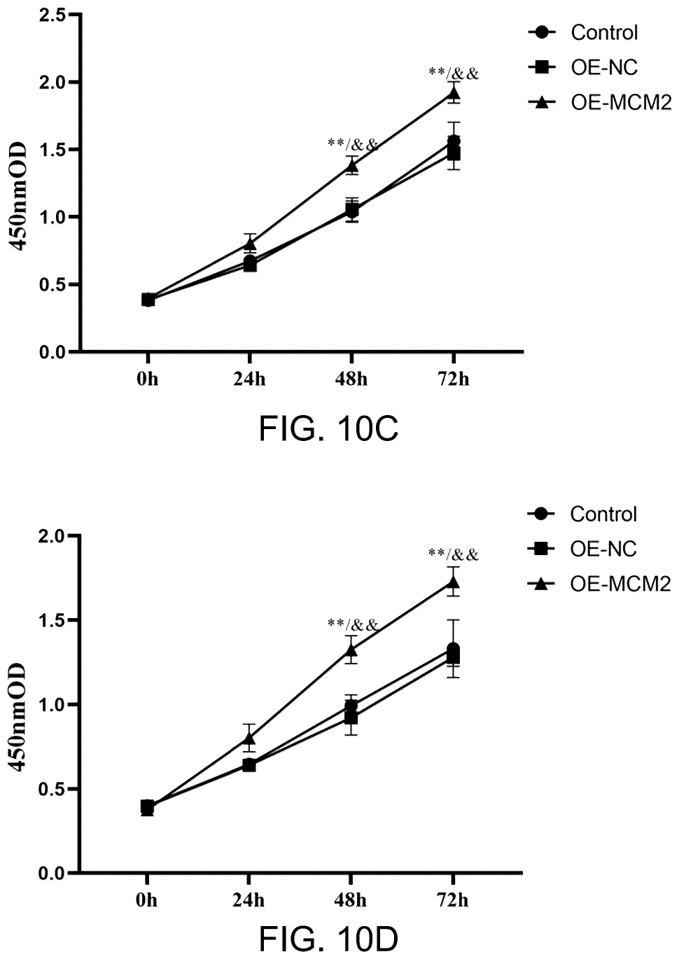

As shown in FIGS. 10A-10D, knocking down MCM2 reduced the proliferation ability of SKOV3 and Hey cells (FIGS. 10A, 10B). The sh-MCM2 group showed a significant decrease in cell proliferation ability compared to the Control group and sh-NC group (P<0.01); and there was no significant change in the proliferation ability of cells in the Control group and sh-NC group. The experimental results of MCM2 overexpression cells were consistent with those of MCM2 knockdown cells. MCM2 overexpression promoted the proliferation ability of SKOV3 and Hey cells (FIGS. 10C, 10D), and the OE-MCM2 group showed a significant increase compared to the Control group and OE-NC group (P<0.01); and there was no significant change in the proliferation ability of cells in the Control group and OE-NC group. This experiment demonstrated that knocking down MCM2 inhibited the proliferation of the ovarian cancer cell; overexpression of MCM2 promoted the proliferation of the ovarian cancer cell; and negative viruses had no significant effect on cell proliferation. Compared to the Control, ** P<0.01; and compared with the sh-NC group or OE-NC group, &&P<0.01.

### 2. The Effect of MCM2 Gene on the Proliferation and Colony Formation of an Ovarian Cancer Cell

SKOV3 and Hey ovarian cancer cells in logarithmic growth phase were knocked down with MCM2 (Control group, sh-NC group, sh-MCM2 group) and overexpressed with MCM2 (Control group, OE-NC group, OE-MCM2 group). The original culture medium was discarded, washed with PBS buffer, and digested with 0.1% trypsin. A corresponding culture medium was added to prepare a cell suspension. Cells were counted using a hemocytometer and a cell concentration was quantified at 10³ cells/mL. 1500 μL of each well was inoculated into a 6 well plate, with 3 parallel wells in each group, and incubated in a 37° C. incubator containing 5% CO₂ for 12 days. The medium was changed every three days. After the cultivation time was over, the six well plate was taken out, and the original culture medium was poured out. An appropriate amount of 4% paraformaldehyde was added to each well and fixed for 30 minutes. The plate was washed twice with a PBS buffer after pouring out the paraformaldehyde, an appropriate amount of 1% crystal violet solution was added for staining for 10 minutes, the crystal violet solution was poured out, and the plate was washed twice with PBS. Photos were taken, and the number of cell clones formed was counted.

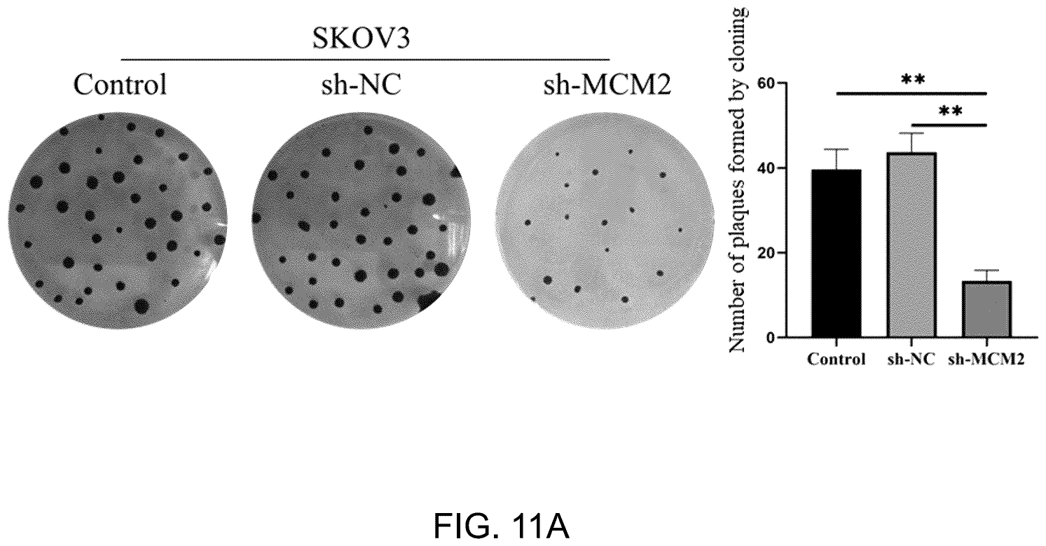

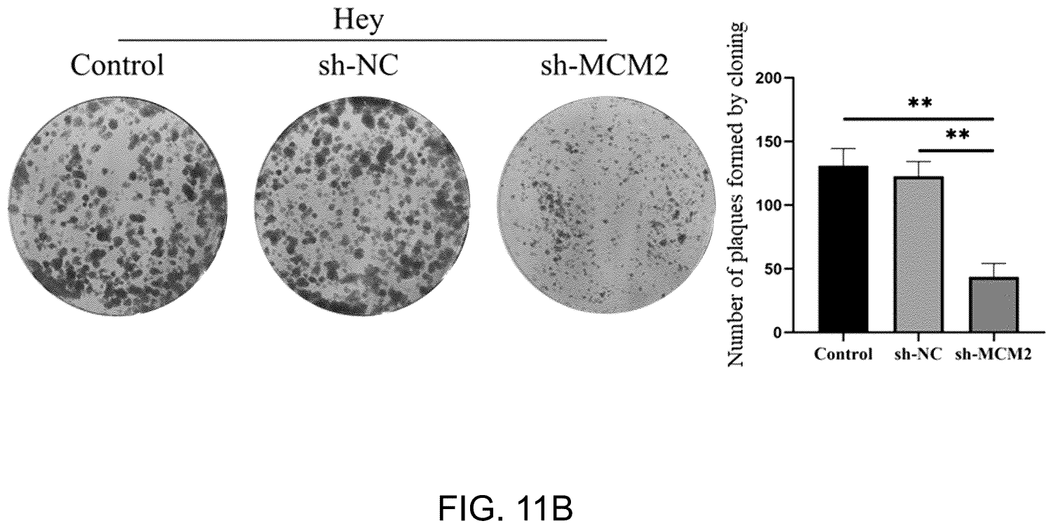

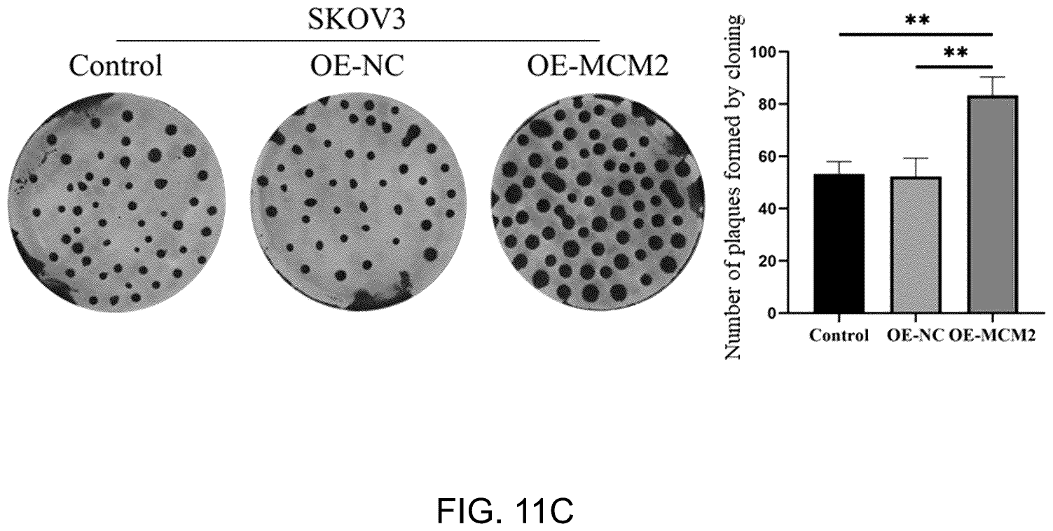

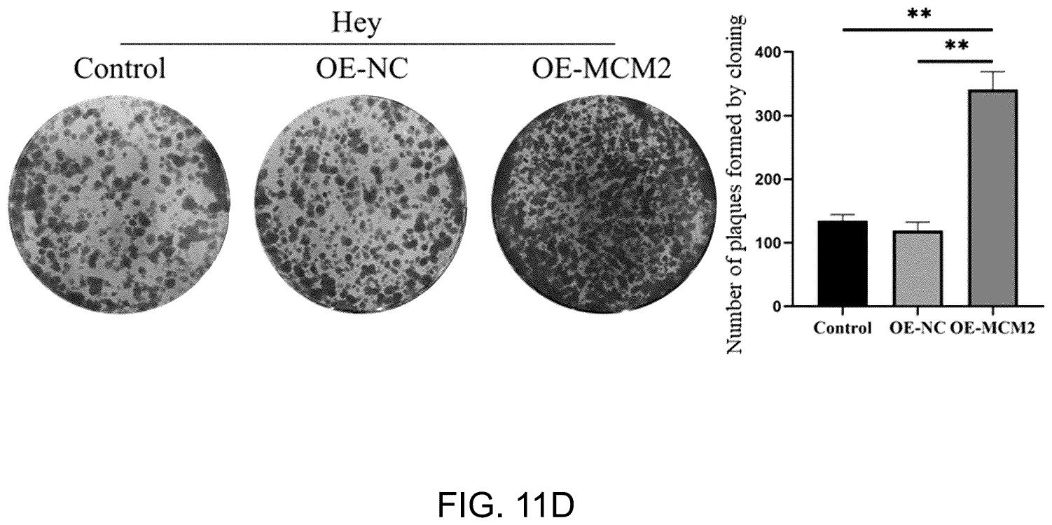

As shown in FIGS. 11A-11D, knocking down MCM2 reduced the colony forming and proliferation abilities of SKOV3 and Hey cells (FIGS. 11A, 11B). The number of plaques formed by cloning in the sh-MCM2 group was significantly reduced compared to the Control group and sh-NC group (P<0.01); and there was no significant change in the colony forming ability and proliferation ability of cells in the Control group and sh-NC group. MCM2 overexpression promoted the colony formation and proliferation abilities of SKOV3 and Hey cells (FIGS. 11C, 11D). The number of plaques formed by cloning in the OE-MCM2 group was significantly increased compared to the Control group and OE-NC group (P<0.01); and there was no significant change in the colony forming ability and proliferation ability of cells in the Control group and OE-NC group. This experiment demonstrated that knocking down MCM2 inhibited the proliferation of the ovarian cancer cell and colony formation; overexpression of MCM2 promoted the proliferation of the ovarian cancer cell and colony formation; and negative viruses had no significant effect on cell proliferation and colony formation.

### 3. The Effect of MCM2 Gene on the Migration and Invasion of an Ovarian Cancer Cell

The night before collecting cells, small chambers were placed in a 24 well plate. A matrix gel was taken out from the −20° C. refrigerator and placed to melt naturally on ice. The thawed matrix gel was mixed with serum-free medium in a ratio of 1:5, and 100 μL of the mixture was added to each chamber. After preparation, the 24 well plate was placed in the incubator overnight. The next day, SKOV3 and Hey ovarian cancer cells in logarithmic growth phase were knocked down with MCM2 (Control group, sh-NC group, sh-MCM2 group) and overexpressed with MCM2 (Control group, OE-NC group, OE-MCM2 group). The original culture medium was discarded, washed with PBS buffer, and digested with 0.1% trypsin. A corresponding serum-free culture medium was added to prepare a cell suspension. Cells were counted using a hemocytometer and the cell concentration was quantified at 5×10⁴ cells/mL. The 24 well plate was taken out and placed in a small chamber for a migration experiment and the 24 well plate with the matrix gel for an invasion experiment. 200 μL of a corresponding cell suspension was added to an upper chamber, and 700 μL of a corresponding culture medium containing 10% FBS was added to a lower chamber. The 24 well plate was further placed in an incubator for cultivation. The 24 well plate for the migration experiment was cultured for 24 hours before being taken out, while the 24 well plate for the invasion experiment was cultured for 48 hours before being taken out. The culture medium in the small chamber was poured out. The inside of the small chamber was cleaned with a cotton swab, and washed twice with the PBS buffer. The small chamber was fixed in 4% paraformaldehyde for 30 minutes, and stained in 1% crystal violet solution for 10 minutes after fixation. The small chamber was taken out, washed twice with the PBS buffer, dried with an absorbent paper, observed and counted under a microscope.

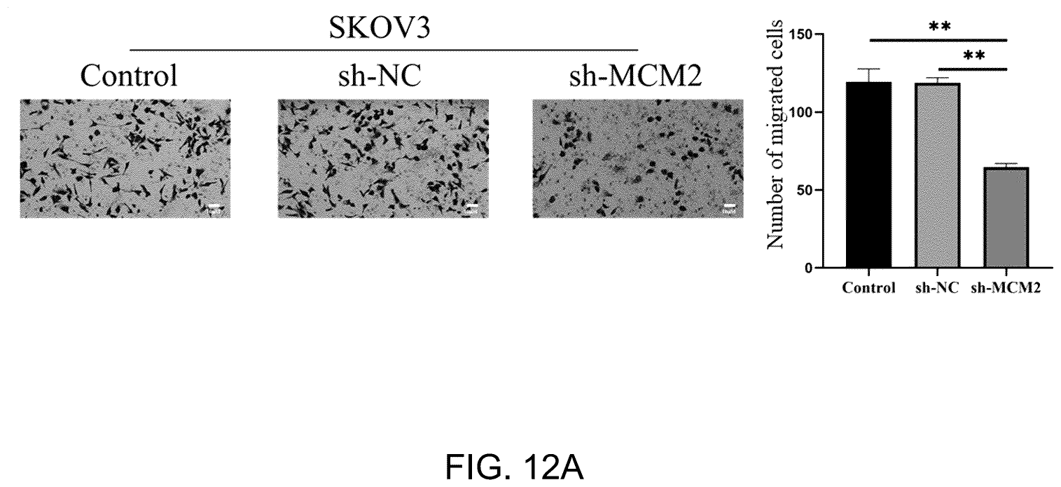

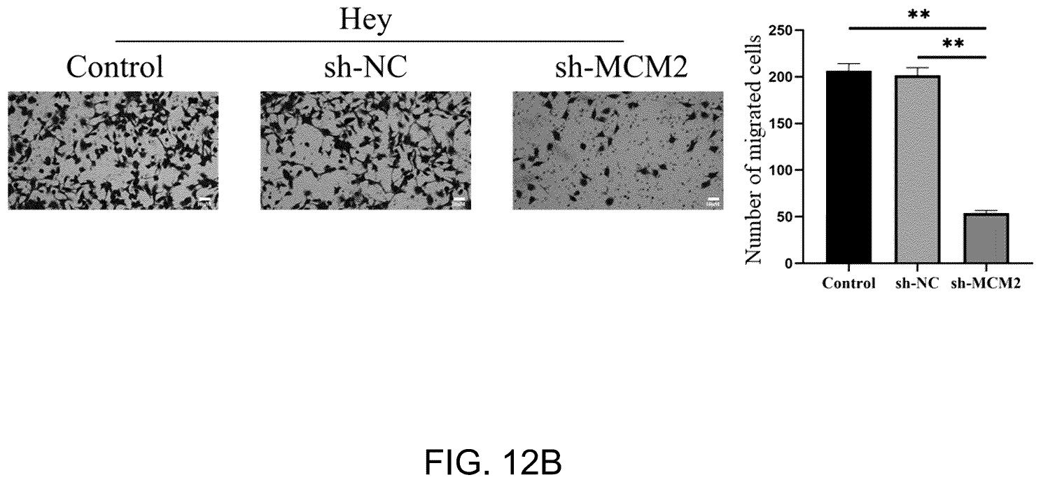

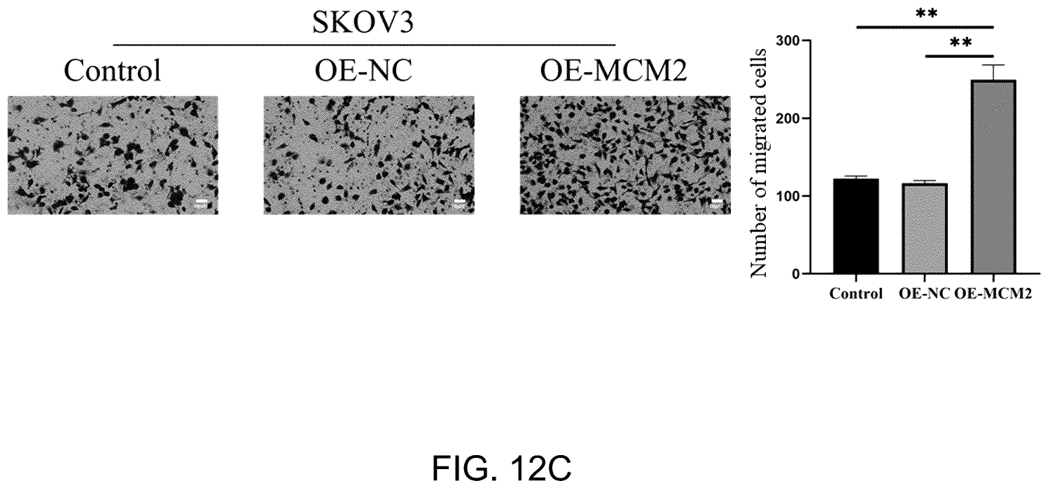

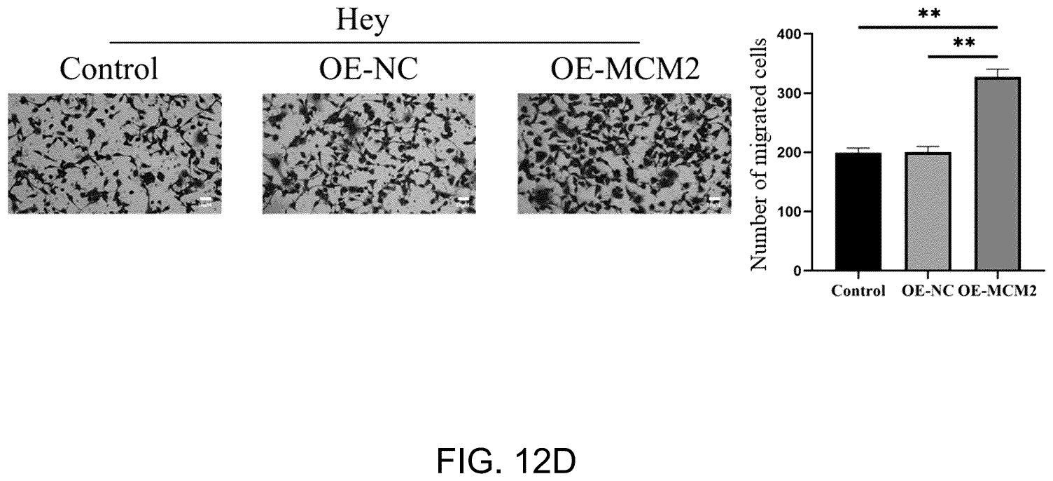

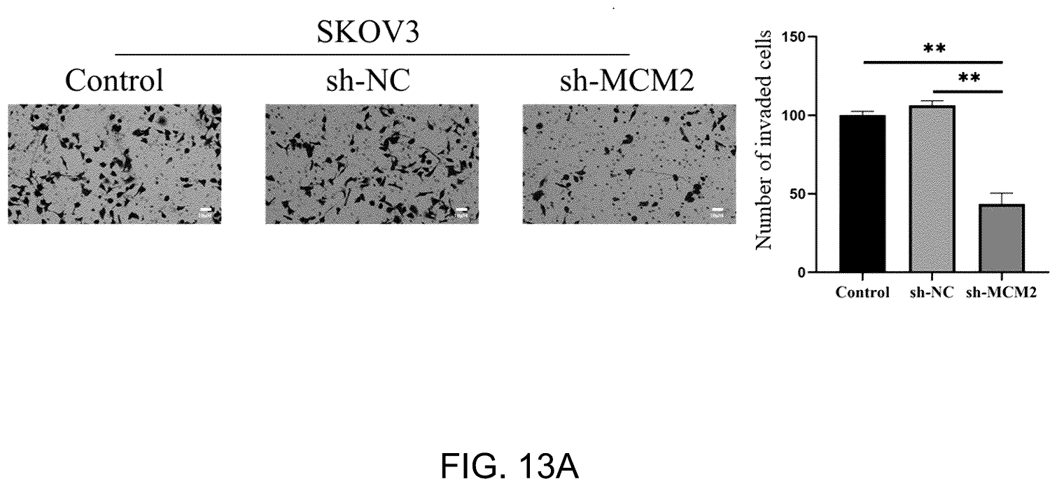

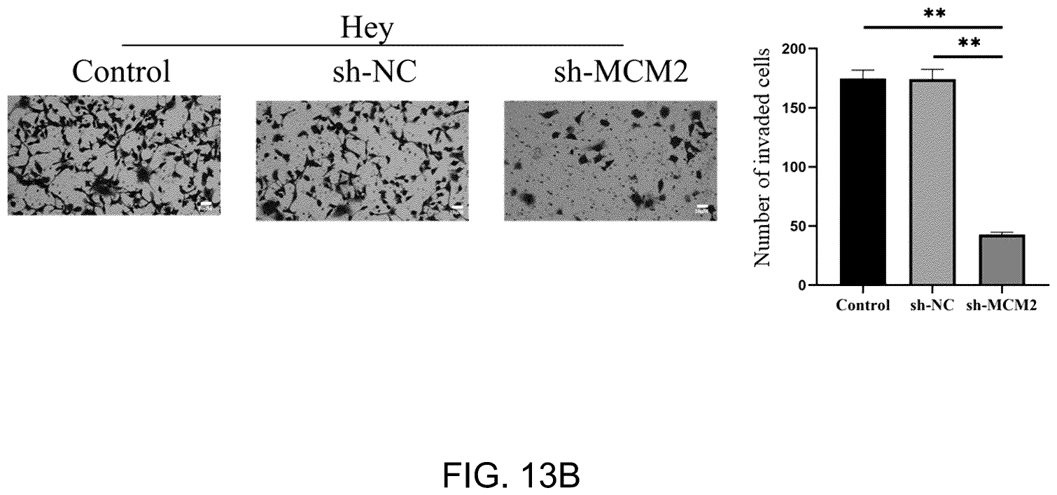

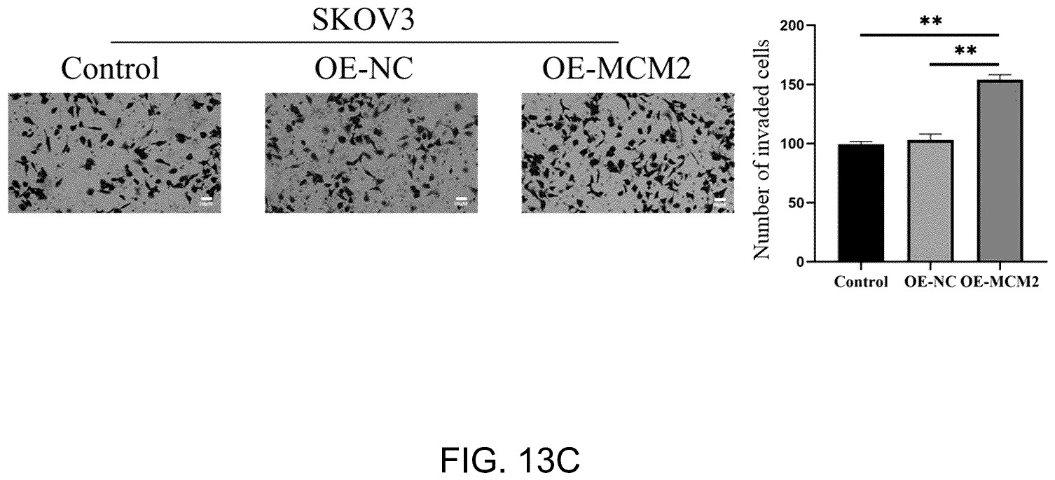

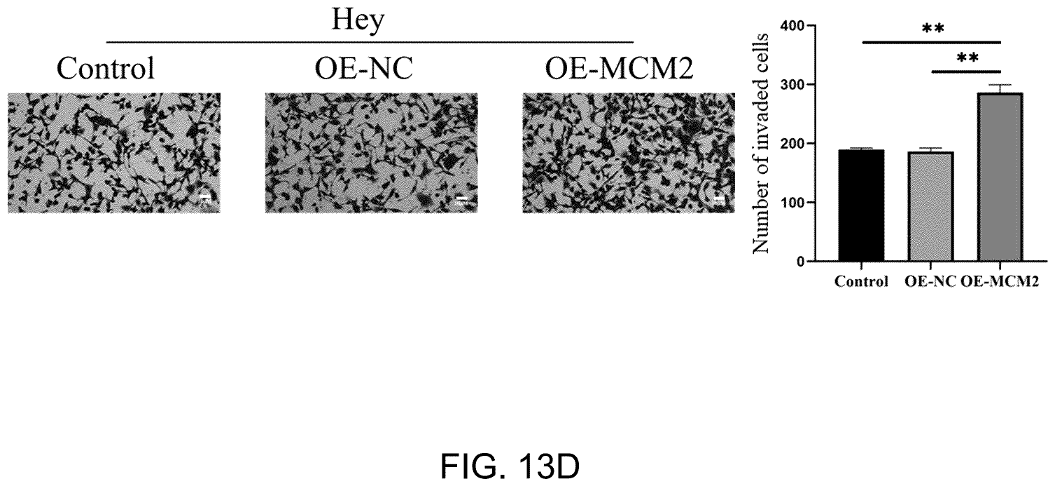

As shown in FIGS. 12A-12D and 13A-13D (with a scale of 10 μM), after knocking down MCM2, the migration (FIGS. 12A, 12B) and invasion (FIGS. 13A, 13B) abilities of the SKOV3 and Hey cells were significantly reduced. The number of the migrating and invading cells in the sh-MCM2 group was significantly lower than that in the Control group and sh-NC group (P<0.01); and there was no significant change in the number of the migrating and invading cells in the Control group and sh-NC group. After overexpression of MCM2, the migration (FIGS. 12C, 12D) and invasion (FIGS. 13C, 13D) abilities of the SKOV3 and Hey cells were significantly increased. The number of the migrating and invading cells in the OE-MCM2 group was significantly higher than that in the Control group and OE-NC group (P<0.01); and there was no significant change in the number of the migrating and invading cells between the Control group and OE-NC group cells. This experiment demonstrated that knocking down MCM2 inhibited the migration and invasion of the ovarian cancer cell; overexpression of MCM2 promoted the migration and invasion of the ovarian cancer cell; and negative viruses had no significant effect on cell migration and invasion.

### Example 4 CA and BDMC Use MCM2 as a Target for Inhibiting Ovarian Cancer

### 1. Molecular Docking Experiment

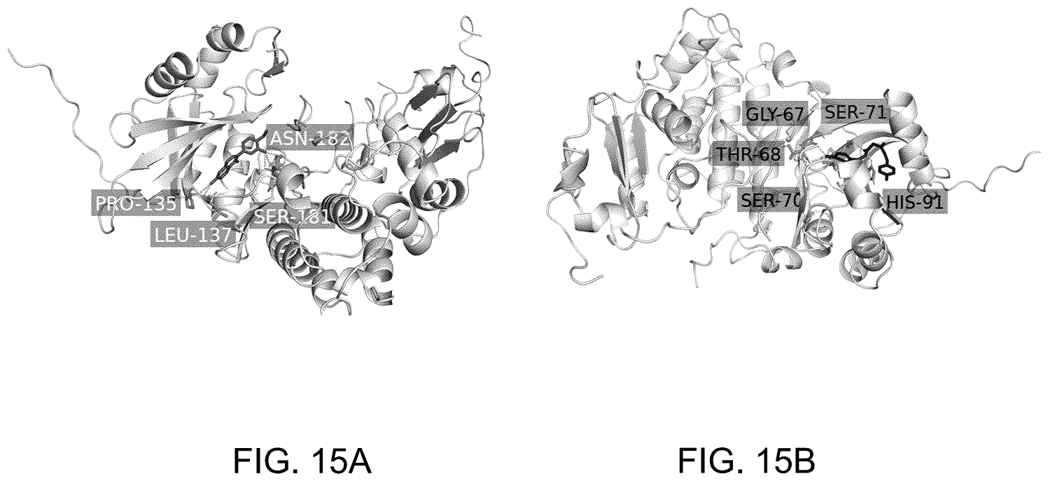

The results of molecular docking were visualized using a Pymol software (FIGS. 15A-15B). In FIGS. 15A-15B, A was the docking diagram of CA and MCM2 molecules; B was the docking diagram of BDMC and MCM2 molecules; the gray structure represented MCM2 protein, red represented small molecule ligand CA, blue represented small molecule ligand BDMC, and green represented amino acid residues connected between the protein and components. The visualization results showed that CA and BDMC could tightly bind to multiple amino acids on the MCM2 protein through a hydrogen bond.

### 2. The Effects of CA and BDMC on the Expression of MCM2 Protein in an Ovarian Cancer Cell

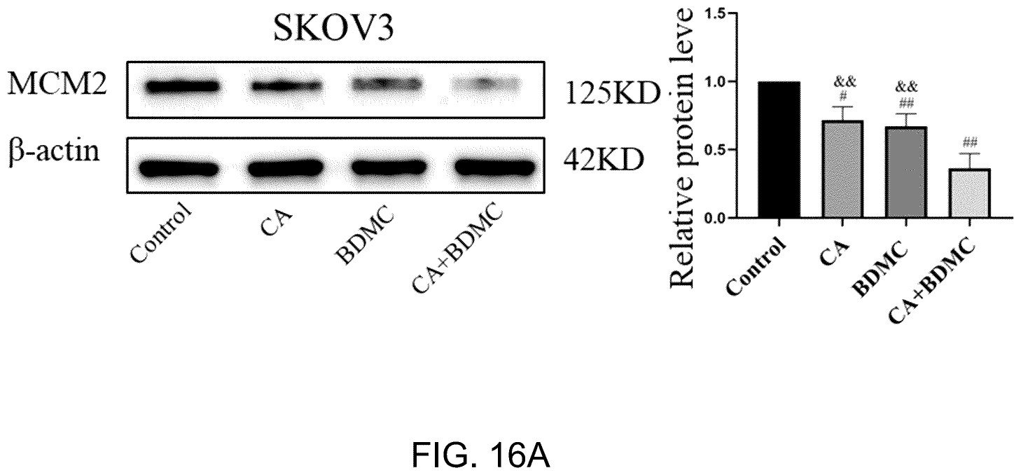

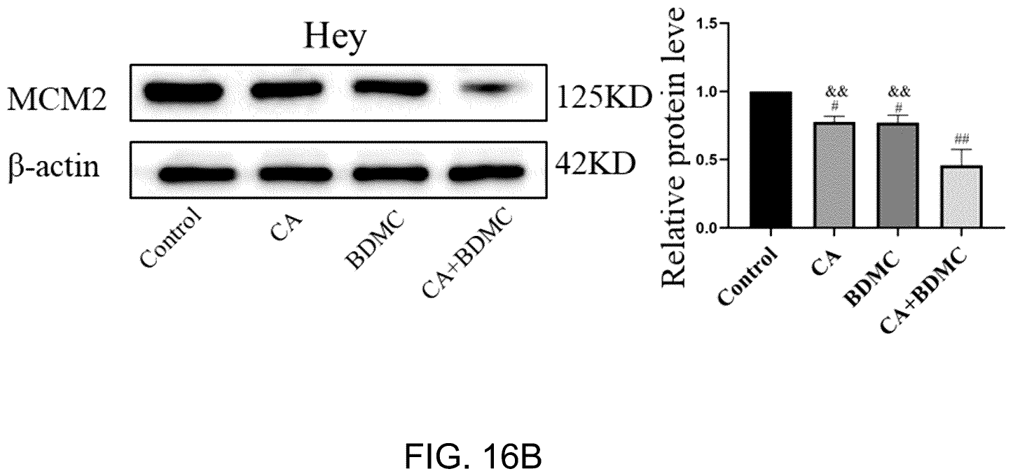

The effect of intervention on MCM2 protein expression in the SKOV3 and Hey ovarian cancer cells for 24 hours in the CA group using 100 μmol/L CA, the BDMC group using 6.25 μmol/L BDMC, and the CA+BDMC group using 100 μmol/L CA+6.25 μmol/L BDMC in Example 1 was shown in FIGS. 16A-16B, wherein A represented the expression of MCM2 protein in SKOV3 cells; B represented the expression of MCM2 protein in Hey cells compared to the Control group, #P<0.05, ##P<0.01; and compared with the CA+BDMC group, &P<0.05, &&P<0.01. In both types of the ovarian cancer cells, the expression of the MCM2 protein was significantly decreased compared to the Control group (P<0.05, P<0.01). At the same time, the expression of the MCM2 protein in the CA+BDMC group was significantly decreased compared to both the CA group and the BDMC group administered separately (P<0.01). This experiment showed that CA, BDMC, and combined administration intervention could reduce the expression of the MCM2 protein in the ovarian cancer cell, and the combined administration group was more effective than each single drug group.

### 3. CA, BDMC, and Combined Administration Affected Ovarian Cancer Cell Proliferation and Colony Formation Ability Through MCM2

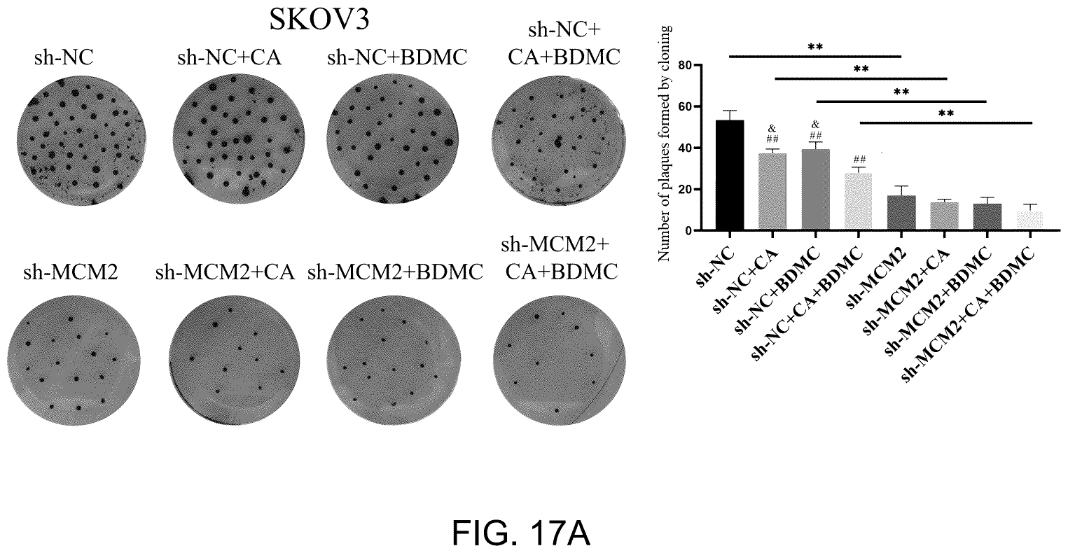

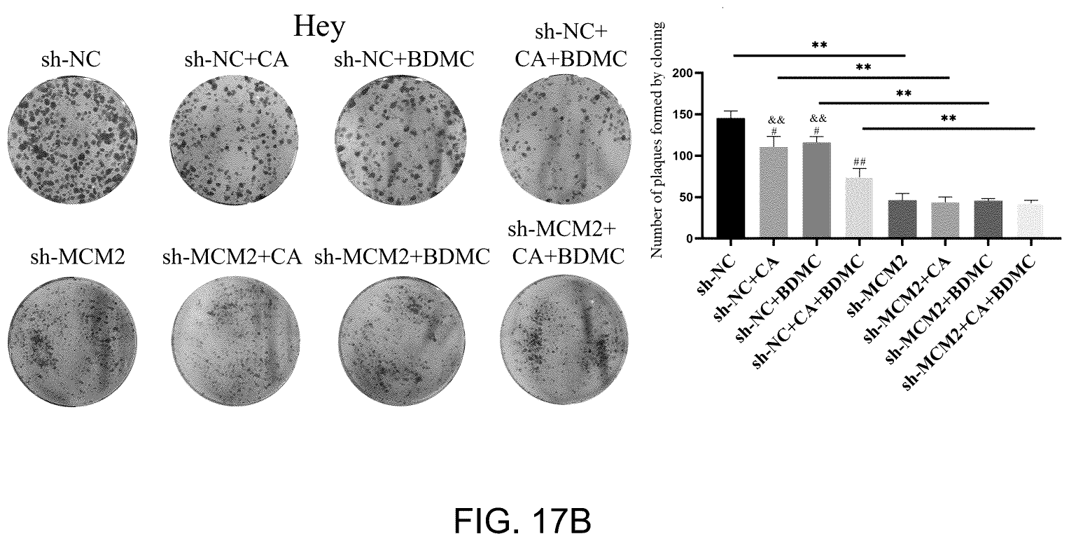

After knocking down MCM2 on the SKOV3 and Hey ovarian cancer cells and intervening with CA, BDMC, and combination administration, the results were shown in FIGS. 17A-17B. In FIGS. 17A-17B, A represented the formation of SKOV3 cell clones; and B represented the formation of Hey cell clones. Compared with the sh-NC group, #P<0.05, ##P<0.01; compared with the sh-NC+CA+BDMC group, &P<0.05, &&P<0.01; and ** P<0.01.

From FIGS. 17A-17B, it could be seen that the lentivirus empty vector infected sh-NC cells of SKOV3 and Hey ovarian cancer cells reduced their colony formation and proliferation abilities after intervention with CA, BDMC, and combined administration. The number of plaques formed by cloning in the sh-NC+CA group, sh-NC+BDMC group, and sh-NC+CA+BDMC group of the two types of cells was significantly reduced compared to the sh-NC group (P<0.05, P<0.01). The number of plaques formed by cloning in the sh-NC+CA+BDMC group was significantly reduced compared to the sh-NC+CA group and the sh-NC+BDMC group in the combined administration group (P<0.05, P<0.01). After knocking down MCM2, there was no significant change in the colony formation and proliferation abilities of cells after intervention with CA, BDMC, and combined administration. The number of plaques formed by cloning in the sh-MCM2+CA group, sh-MCM2+BDMC group, and sh-MCM2+CA+BDMC group of the two types of cells showed no significant change compared to the sh-MCM2 group. The number of plaques formed by cloning in the sh-MCM2+CA+BDMC group showed no significant change compared to the sh-MCM2+CA group and sh-MCM2+BDMC group in the combined administration group. Meanwhile, under the same administration treatment conditions, the number of plaques formed by cloning in the sh-MCM2 cell treatment group was significantly reduced compared to the sh-NC cell treatment group (P<0.01).

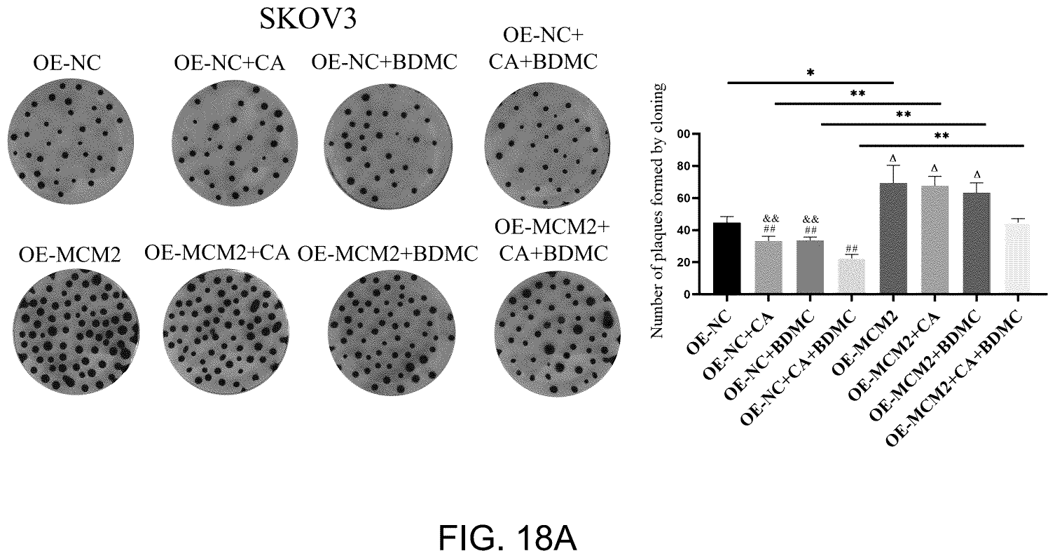

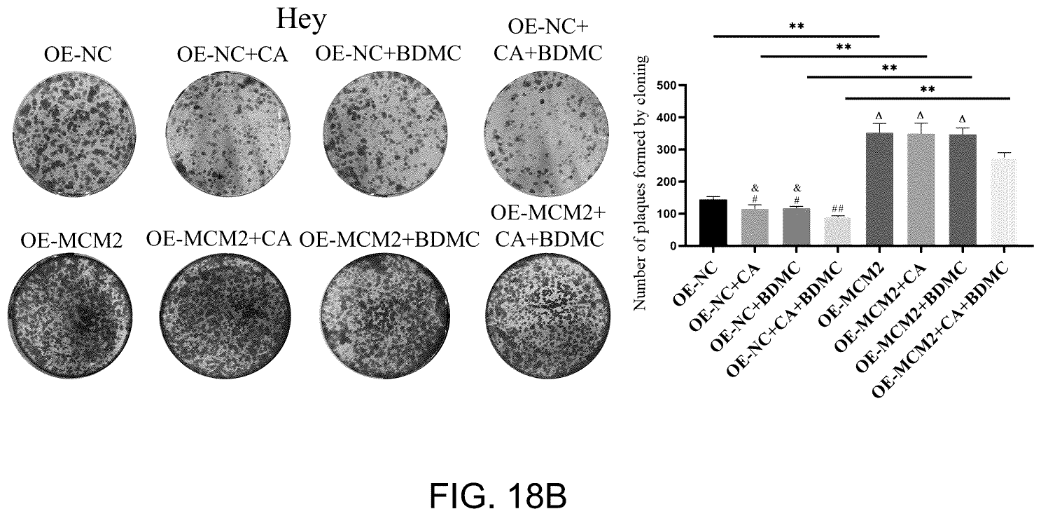

The results of the administration intervention experiment on cells overexpressing MCM2 were shown in FIGS. 18A-18B. In FIGS. 18A-18B, A represented the formation of SKOV3 cell clones; and B represented the formation of Hey cell clones. Compared with the OE-NC group, #P<0.05, ##P<0.01; compared with the OE-NC+CA+BDMC group, &P<0.05, &&P<0.01; and *P<0.05; and compared with the OE-MCM2+CA+BDMC group, ΔP<0.05, ΔΔP<0.01; and *P<0.05, ** P<0.01.

From FIGS. 18A-18B, it could be seen that the lentivirus empty vector infected OE-NC cells of SKOV3 and Hey ovarian cancer cells reduced their colony formation and proliferation abilities after intervention with CA, BDMC, and combined administration. The number of plaques formed by cloning in the OE-NC+CA group, OE-NC+BDMC group, and OE-NC+CA+BDMC group of the two types of cells was significantly reduced compared to the OE-NC group (P<0.05, P<0.01). The number of plaques formed by cloning in the OE-NC+CA+BDMC group was significantly reduced compared to the OE-NC+CA group and the OE-NC+BDMC group in the combined administration group (P<0.05, P<0.01). After overexpression of MCM2, there was no significant change in the colony formation and proliferation abilities of cells after single drug intervention with CA and BDMC. The number of plaques formed by cloning in the OE-MCM2+CA group and OE-MCM2+BDMC group of the two types of cells showed no significant change compared to the OE-MCM2 group. The number of plaques formed by cloning in the OE-MCM2+CA+BDMC group decreased significantly compared to the OE-MCM2 group, OE-MCM2+CA group, and OE-MCM2+BDMC group in the combined administration group (P<0.05, P<0.01). Meanwhile, under the same administration treatment conditions, the number of plaques formed by cloning of OE-MCM2 cells significantly increased compared to OE-NC cells (P<0.05, P<0.01). This experiment showed that CA, BDMC, and combined administration intervention could reduce the colony formation and proliferation abilities of the ovarian cancer cell, and the combined administration group is more effective than each single drug group; and after knocking down and overexpressing MCM2, the drug efficacy was weakened. However, after knocking down MCM2, there was no significant change in the number of plaques formed by cell cloning in each group after intervention with administration. After overexpressing MCM2, only the combined administration group showed efficacy.

### 4. CA, BDMC, and Combined Administration Affected Ovarian Cancer Cell Migration and Invasion Through MCM2

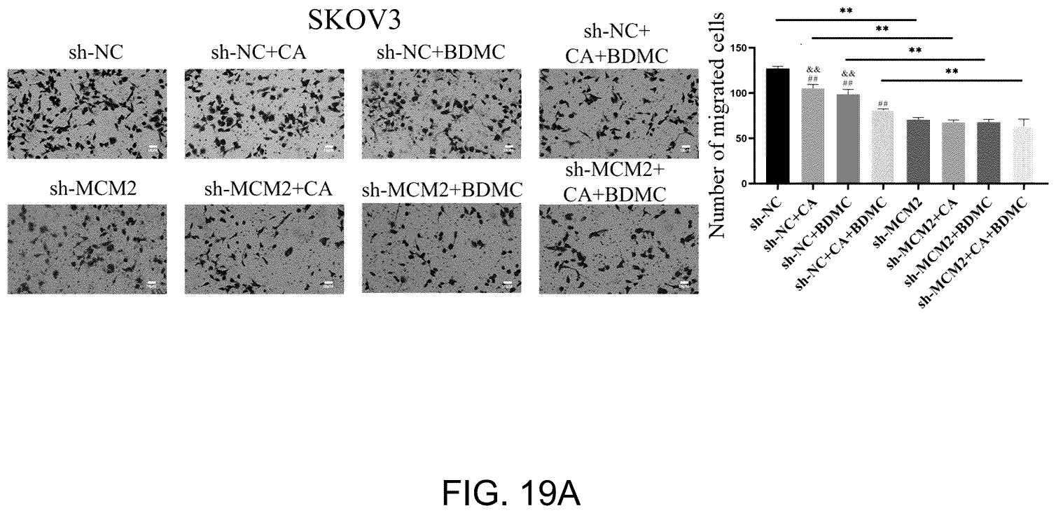

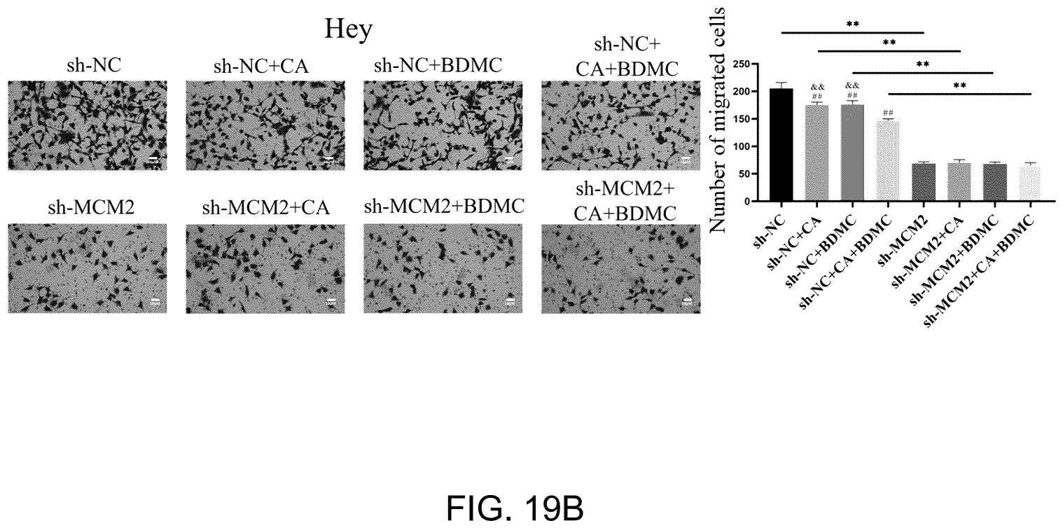

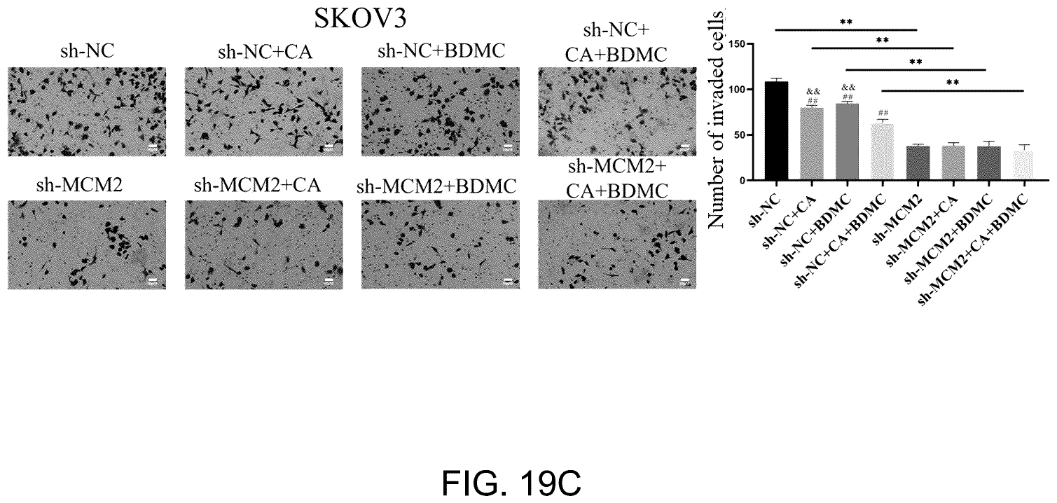

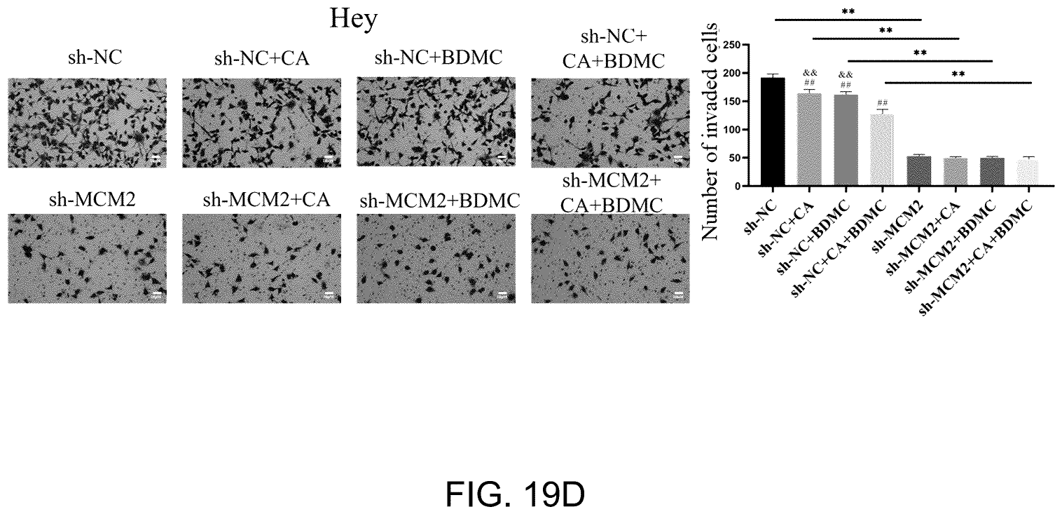

The impact of CA, BDMC, and combined administration intervention on cell migration after knocking down MCM2 in SKOV3 and Hey ovarian cancer cells was shown in FIGS. 19A-19D. The scale in FIGS. 19A-19D was 10 μM; A represented the migration of SKOV3 cells; B represented the migration of Hey cells; C represented the invasion of SKOV3 cells; and D represented the invasion of Hey cells. Compared with the sh-NC group, ##P<0.01; compared with the sh-NC+CA+BDMC group, &&P<0.01; and ** P<0.01.

As shown in FIGS. 19A-19D, it could be seen that the lentivirus empty vector infected sh-NC cells of SKOV3 and Hey ovarian cancer cells affected the migration and invasion abilities after intervention with CA, BDMC, and combined administration. The number of the migrating and invading cells in the sh-NC+CA group, sh-NC+BDMC group, and sh-NC+CA+BDMC group of the two types of cells was significantly reduced compared to the sh-NC group (P<0.01). The number of the migrating and invading cells in the sh-NC+CA+BDMC group was significantly reduced compared to the sh-NC+CA group and the sh-NC+BDMC group in the combined administration group (P<0.01). After knocking down MCM2, there was no significant change in the number of the migrating and invading cells after intervention with CA, BDMC, and combined administration. The number of the migrating and invading cells in the sh-MCM2+CA group, sh-MCM2+BDMC group, and sh-MCM2+CA+BDMC group of the two types of cells showed no significant change compared to the sh-MCM2 group. The number of the migrating and invading cells in the sh-MCM2+CA+BDMC group showed no significant change compared to the sh-MCM2+CA group and sh-MCM2+BDMC group in the combined administration group. Meanwhile, under the same administration treatment conditions, the number of the migrating and invading cells in the sh-MCM2 cell treatment group was significantly reduced compared to the sh-NC cell treatment group (P<0.01).

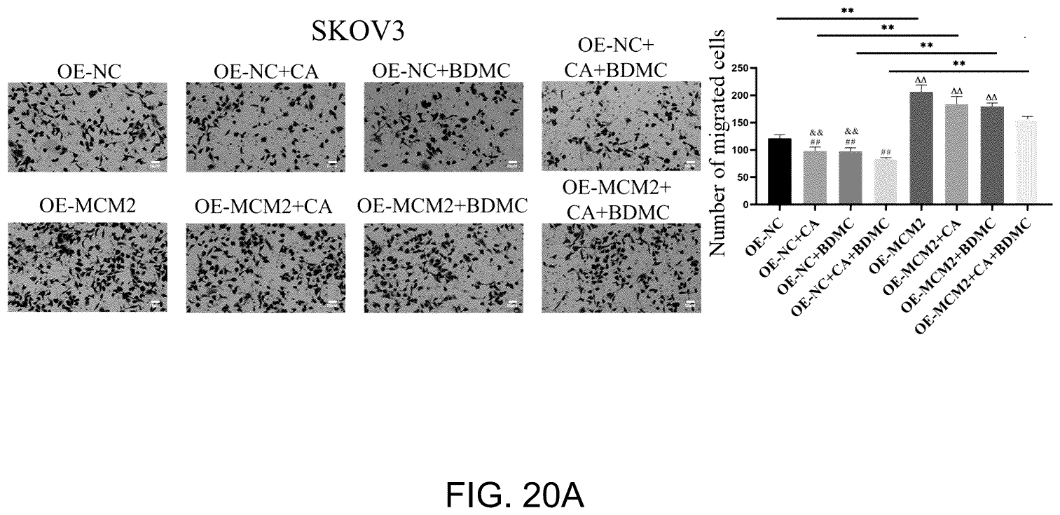

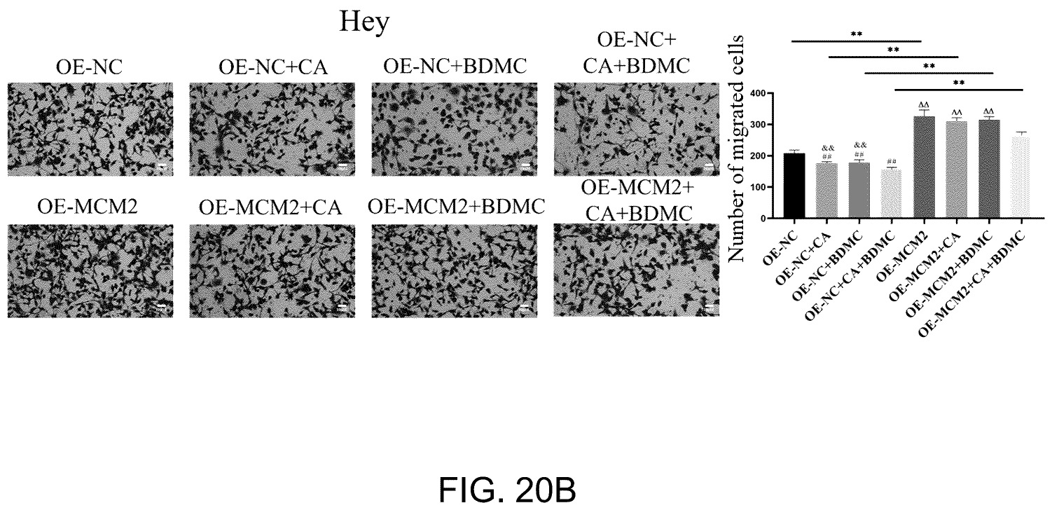

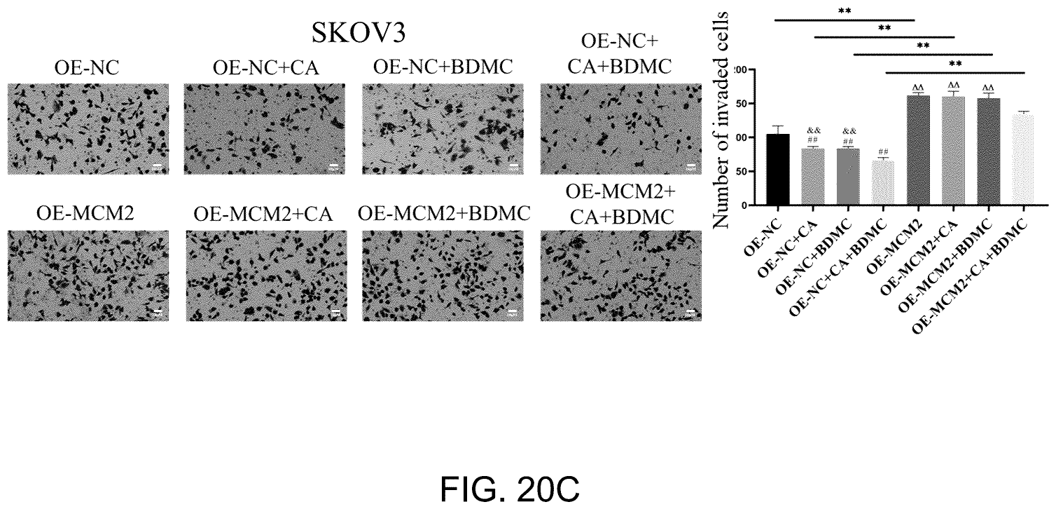

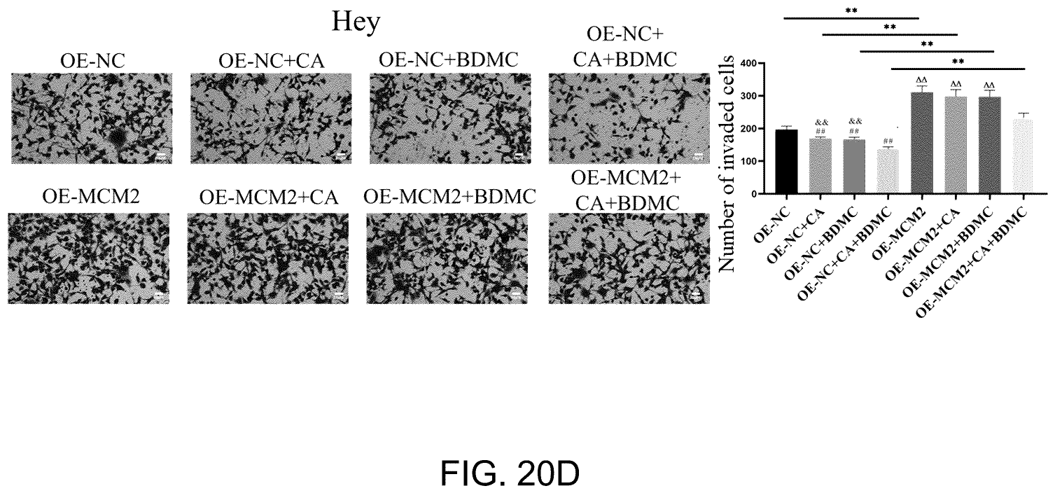

The impact of CA, BDMC, and combined administration intervention on cell migration after overexpression of MCM2 on the SKOV3 and Hey ovarian cancer cells was shown in FIGS. 20A-20D. The scale in FIGS. 20A-20D was 10 μM, A represented the migration of SKOV3 cells; B represented the migration of Hey cells; C represented the invasion of SKOV3 cells; and D represented the invasion of Hey cells. Compared with the OE-NC group, ##P<0.01; compared with the OE-NC+CA+BDMC group, &&P<0.01; and compared with the OE-MCM2+CA+BDMC group, ΔP<0.05, ΔΔP<0.01; and ** P<0.01.

From FIGS. 20A-20D, it could be seen that the lentivirus empty vector infected OE-NC cells of SKOV3 and Hey ovarian cancer cells affected cell migration and invasion abilities after intervention with CA, BDMC, and combined administration. The number of the migrating and invading cells in the OE-NC+CA group, OE-NC+BDMC group, and OE-NC+CA+BDMC group of the two types of cells was significantly reduced compared to the OE-NC group (P<0.01). The number of the migrating and invading cells in the OE-NC+CA+BDMC group was significantly reduced compared to the OE-NC+CA group and the OE-NC+BDMC group in the combined administration group (P<0.01). After overexpression of MCM2, there was no significant change in the migration and invasion abilities of cells after single drug intervention with CA and BDMC. The number of the migrating and invading cells in the OE-MCM2+CA group and OE-MCM2+BDMC group of the two types of cells showed no significant change compared to the OE-MCM2 group. The number of the migrating and invading cells in the OE-MCM2+CA+BDMC group decreased significantly compared to the OE-MCM2 group, OE-MCM2+CA group, and OE-MCM2+BDMC group in the combined administration group (P<0.01). Meanwhile, under the same administration treatment conditions, the number of the migrating and invading cells of OE-MCM2 cells significantly increased compared to OE-NC cells (P<0.01). This experiment showed that CA, BDMC, and combined administration intervention could reduce the migration and invasion abilities of the ovarian cancer cell, and the combined administration group is more effective than each single drug group; and after knocking down and overexpressing MCM2, the drug efficacy was weakened. However, after knocking down MCM2, there was no significant change in the number of the migrating and invading cells in each group after intervention with administration. After overexpressing MCM2, only the combined administration group showed efficacy.

### Example 5 MCM2 Gene as a Tumor Pharmaceutical Target for Inhibiting Tumor Cells

### 1. The Effect of MCM2 Gene on Cell Cycle

SKOV3 and Hey ovarian cancer cells in logarithmic growth phase were knocked down with MCM2 (Control group, sh-NC group, sh-MCM2 group) and overexpressed with MCM2 (Control group, OE-NC group, OE-MCM2 group). The original culture medium was discarded, washed with PBS buffer, and digested with 0.1% trypsin. A corresponding culture medium was added to prepare a cell suspension. Cells were counted using a hemocytometer and a cell concentration was quantified at 5×10⁴ cells/mL. 1500 μL of each well was inoculated into a 6 well plate, with 3 parallel wells in each group, and incubated in a 37° C. incubator containing 5% CO₂ for 24 hours. After the cultivation time was over, the six well plate was taken out and aspirated the original culture medium from the wells for later use. The PBS buffer was added to gently wash the cells three times. 500 μL of 0.1% trypsin was added to each well to wait for the cells to be blown off with a pipette, then the original culture medium from the wells was added to terminate digestion. A pipette was used to gently blow off all the cells on the wall of the six well plate. The cell suspension was aspirated, transferred to a centrifuge tube, and centrifuged at 4° C. and 1000 rpm for 5 minutes. The supernatant was discarded, and 1 mL of pre-cooled PBS buffer was added to gently resuspend the cells, and transferred to a 1.5 mL sterile centrifuge tube. The mixture was centrifuged at 4° C. and 1000 rpm for 5 minutes, the supernatant was discarded, and the bottom of the centrifuge tube was gently tapped to disperse the cells. 1 mL of pre-cooled 70% ethanol was added, blown and mixed well to form a cell suspension, and placed in a refrigerator at 4° C. for 24 hours. After fixation, a water bath kettle was opened in advance, the temperature was set to 37° C., the centrifugation was performed for 5 minutes at 4° C. and 1000 rpm, and the supernatant was discarded. 1 mL of pre-cooled PBS buffer was added and gently resuspended to wash the cells. The mixture was centrifuged at 4° C. and 1000 rpm for 5 minutes, the supernatant was discarded, and the bottom of the centrifuge tube was gently tapped to disperse the cells. 500 μL of PI staining working solution containing RNase was added to each cell sample to be tested, and placed in a 37° C. dark water bath for 30 minutes. A flow cytometer was used to detect red fluorescence at an excitation wavelength of 488 nm, while also measuring light scattering. A modfit 3.2 version was used for cell DNA cycle analysis.

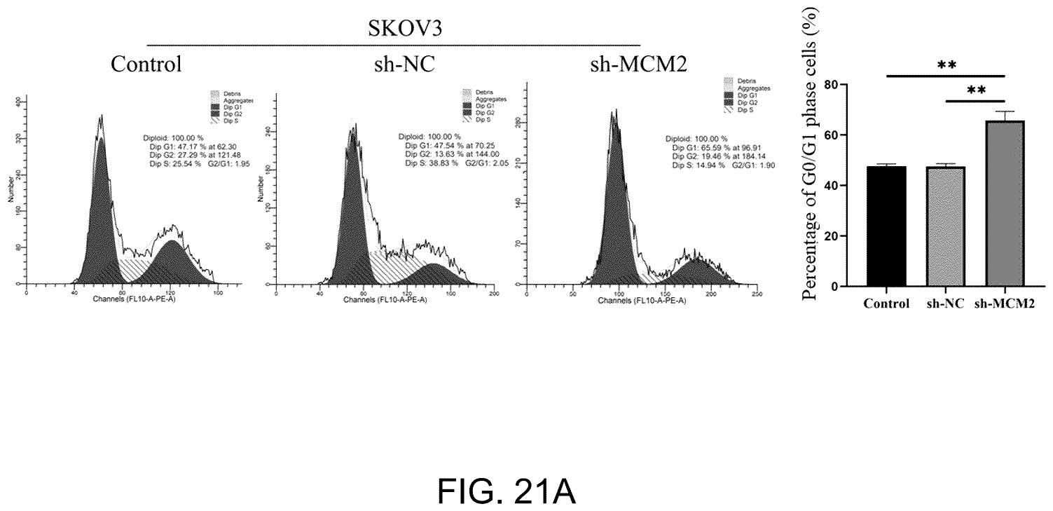

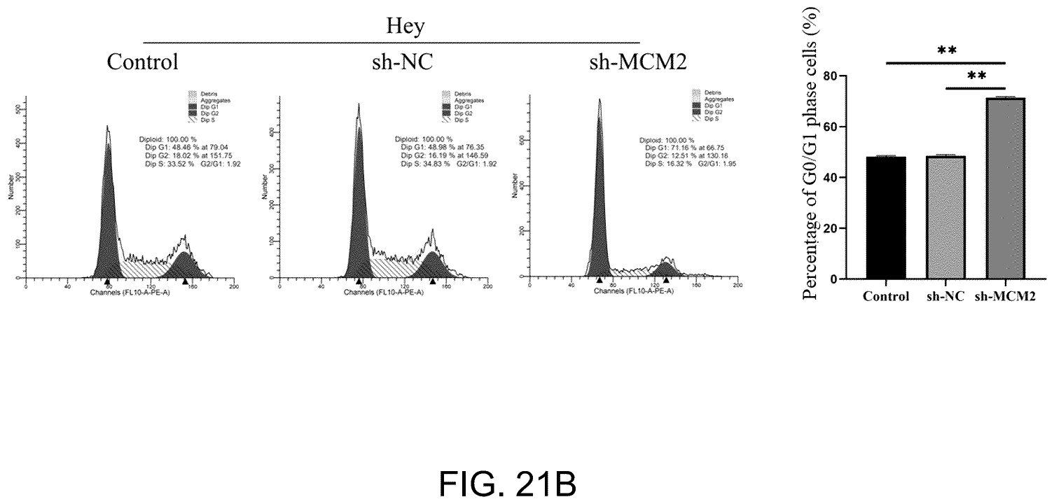

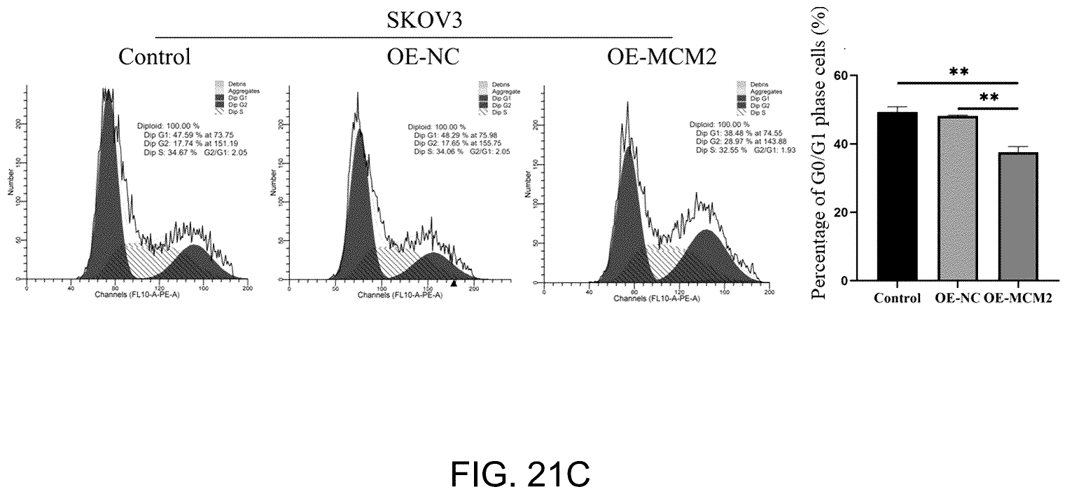

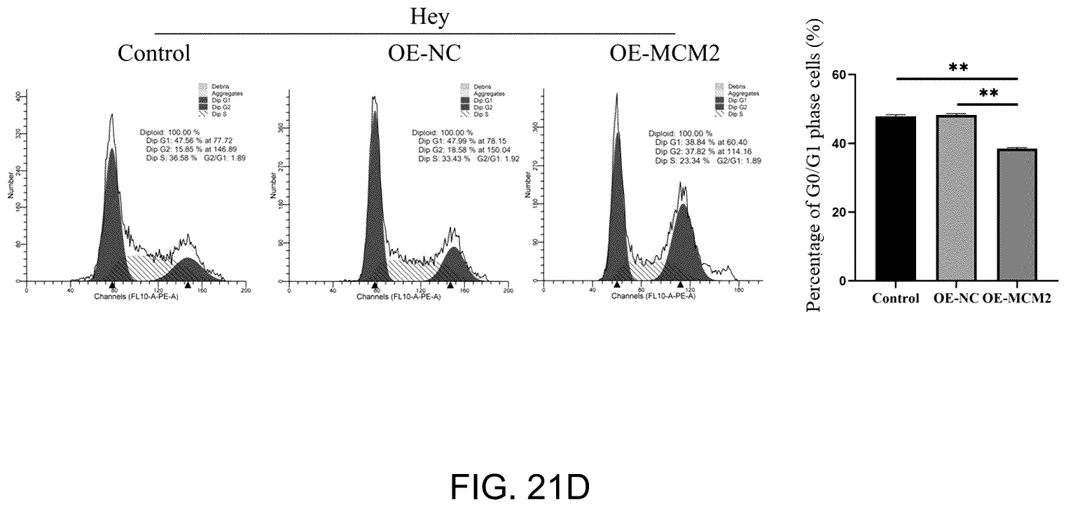

Cell cycle experiments were conducted on SKOV3 and Hey ovarian cancer cells after knocking down and overexpressing MCM2. The results were shown in FIGS. 21A-21D. After knocking down MCM2, the G0/G1 phase ratio of the SKOV3 and Hey cells significantly increased (FIGS. 21A, 21B). The G0/G1 phase ratio of sh-MCM2 group cells was significantly higher than that of the Control group and sh-NC group (P<0.01); and there was no significant change in the cell cycle of the Control group and sh-NC group cells. After overexpression of MCM2, the G0/G1 phase ratio of the SKOV3 and Hey cells significantly increased (FIGS. 21C, 21D), while the G0/G1 phase ratio of the OE-MCM2 group decreased significantly compared to the Control group and OE-NC group (P<0.01); and there was no significant change in the cell cycle of the Control group and OE-NC group cells. This experiment showed that knocking down MCM2 increased the G0/G1 phase ratio in the ovarian cancer cells and arrested the cell cycle in the G0/G1 phase; overexpression of MCM2 led to a decrease in the G0/G1 phase ratio of the ovarian cancer cells and accelerated the cell cycle progression; and negative viruses had no significant effect on the cell cycle.

### 2. The Effect of MCM2 on the Cell Cycle and EMT Related Protein Expression of Ovarian Cancer Cells

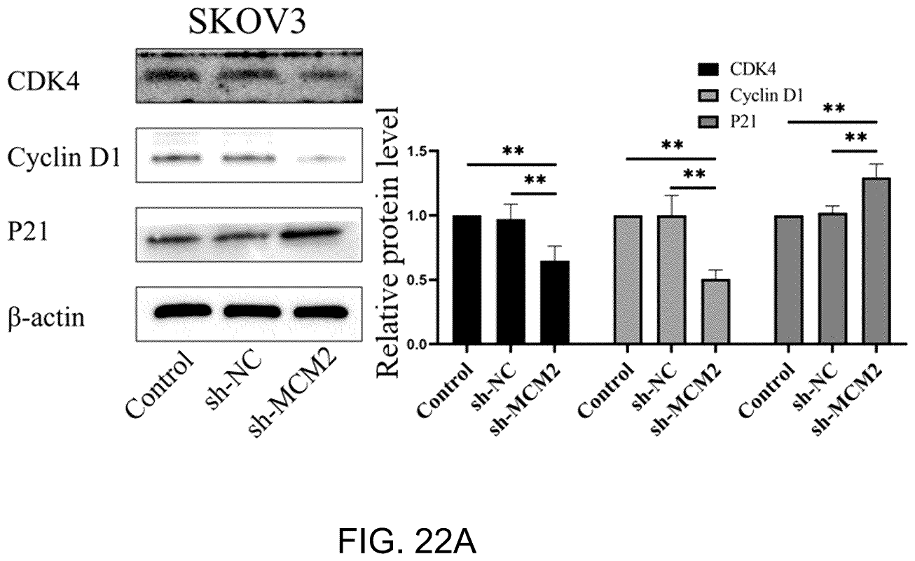

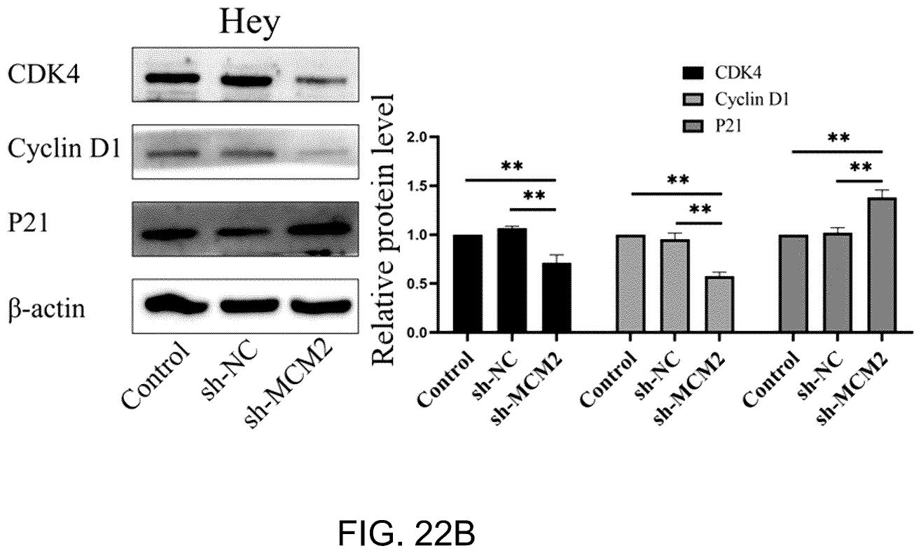

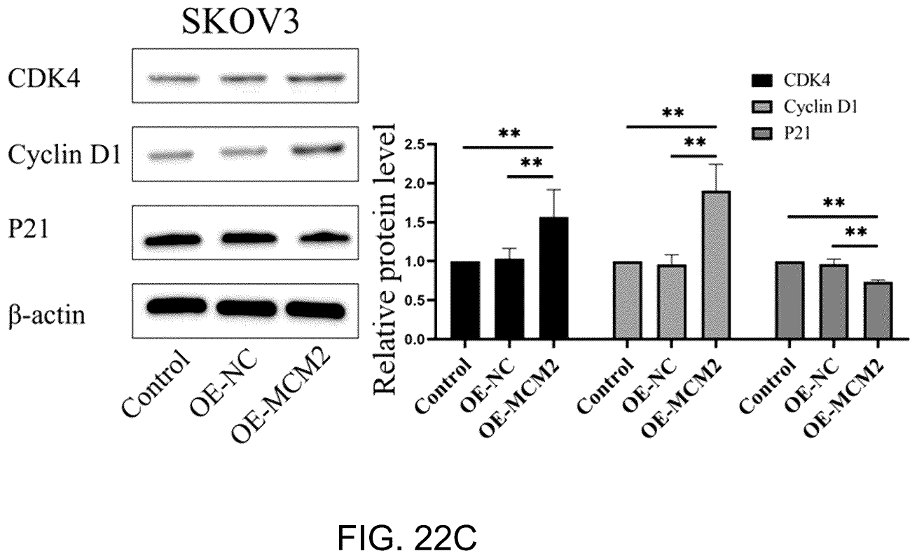

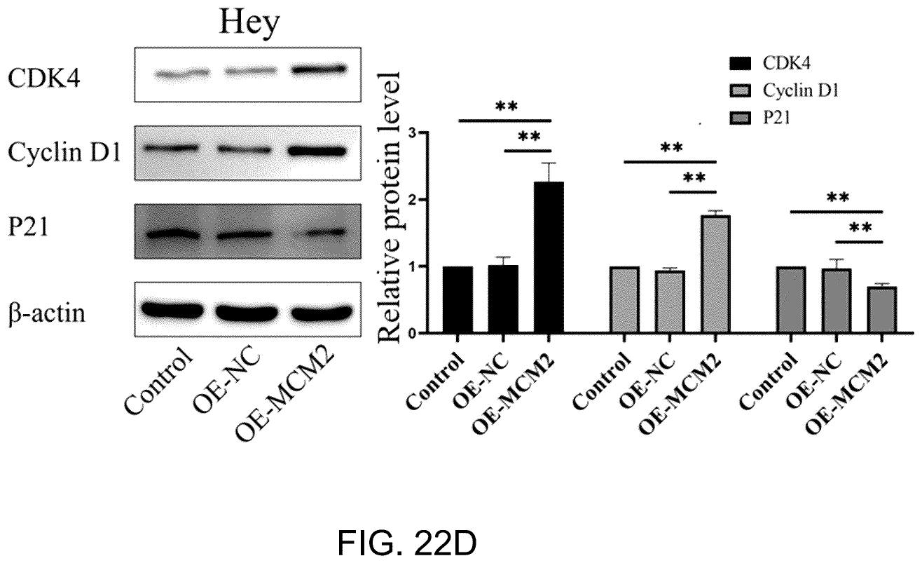

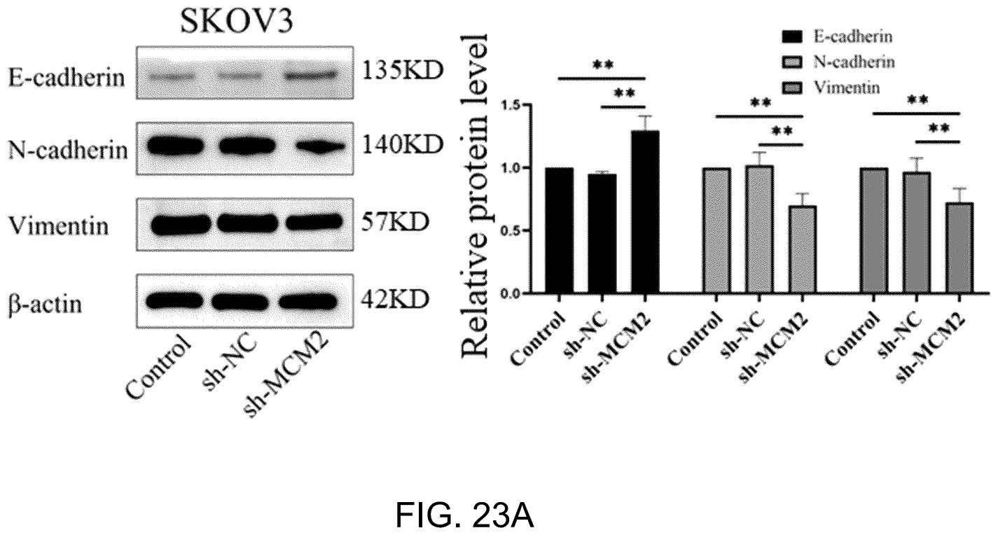

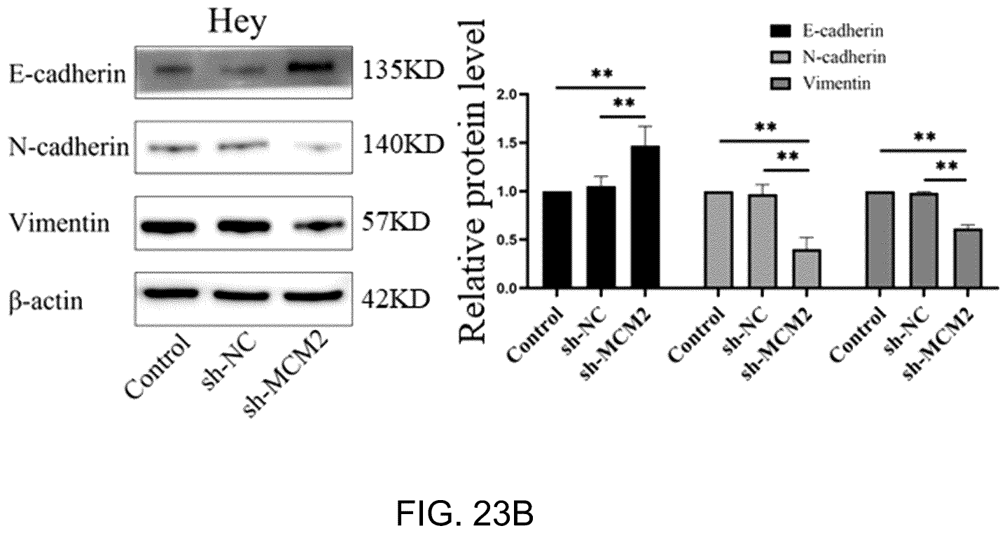

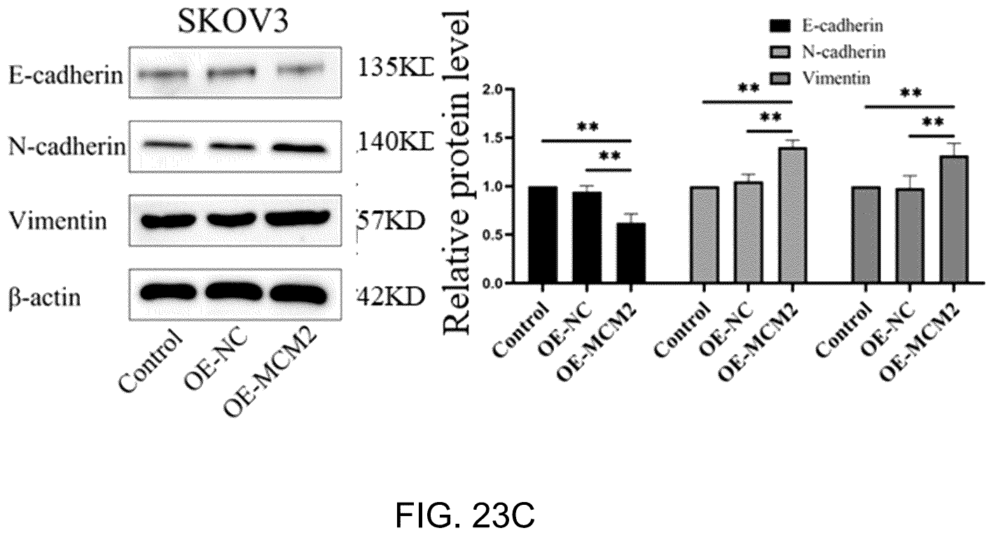

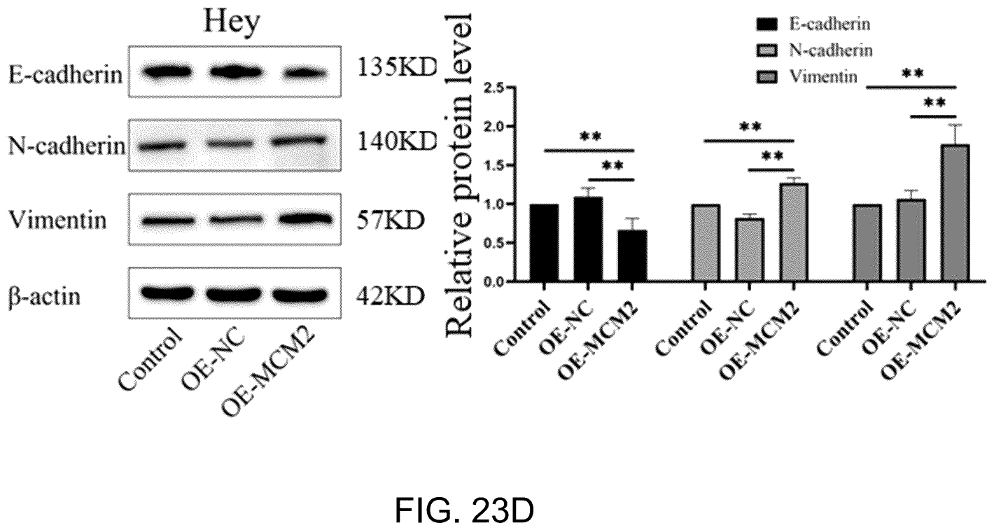

The effects of MCM2 knockdown and overexpression on the cell cycle and EMT related proteins of SKOV3 and Hey cells were shown in FIGS. 22A-22D and 23A-23D. The expression of cell cycle related proteins in SKOV3 and Hey cells after MCM2 knockdown (FIGS. 22A, 22B): the expression of CDK4 and Cyclin D1 in the sh-MCM2 group was significantly decreased compared to the Control group and sh-NC group (P<0.01); and the expression of P21 in the sh-MCM2 group was significantly increased compared to the Control group and sh-NC group (P<0.01), while there was no significant change in the expression of CDK4, Cyclin D1, and P21 in the cells of the Control group and sh-NC group. The expression of EMT related proteins in SKOV3 and Hey cells after MCM2 knockdown (FIGS. 23A, 23B): the E-cadherin expression in the sh-MCM2 group was significantly increased compared to the Control group and sh-NC group (P<0.01); and the expression of N-cadherin and Vimentin in the sh-MCM2 group was significantly decreased compared to the Control group and sh-NC group (P<0.01), while there was no significant change in the expression of E-cadherin, N-cadherin, and Vimentin in the cells of the Control group and sh-NC group.

The expression of cell cycle related proteins in SKOV3 and Hey cells after overexpression of MCM2 (FIGS. 22C, 22D): the expression of CDK4 and Cyclin D1 in the OE-MCM2 group was significantly increased compared to the Control group and OE-NC group (P<0.01); and the expression of P21 in the OE-MCM2 group was significantly decreased compared to the Control group and OE-NC group (P<0.01), while there was no significant change in the expression of CDK4, Cyclin D1, and P21 in the cells of the Control group and OE-NC group. The expression of EMT related proteins in SKOV3 and Hey cells after overexpression of MCM2 (FIGS. 23C, 23D): the expression of E-cadherin in the OE-MCM2 group was significantly decreased compared to the Control group and sh-NC group (P<0.01); and the expression of N-cadherin and Vimentin in the OE-MCM2 group was significantly increased compared to the Control group and OE-NC group (P<0.01), while there was no significant change in the expression of E-cadherin, N-cadherin, and Vimentin in the cells of the Control group and OE-NC group. This experiment demonstrated that knocking down and overexpression of MCM2 could affect the cell cycle and expression of EMT related proteins in the ovarian cancer cells; and negative viruses had no significant effect on the expression of cell cycle proteins and EMT related proteins.

### 1. CA, BDMC, and Combined Administration Affect the Cell Cycle of Ovarian Cancer Cells Through MCM2

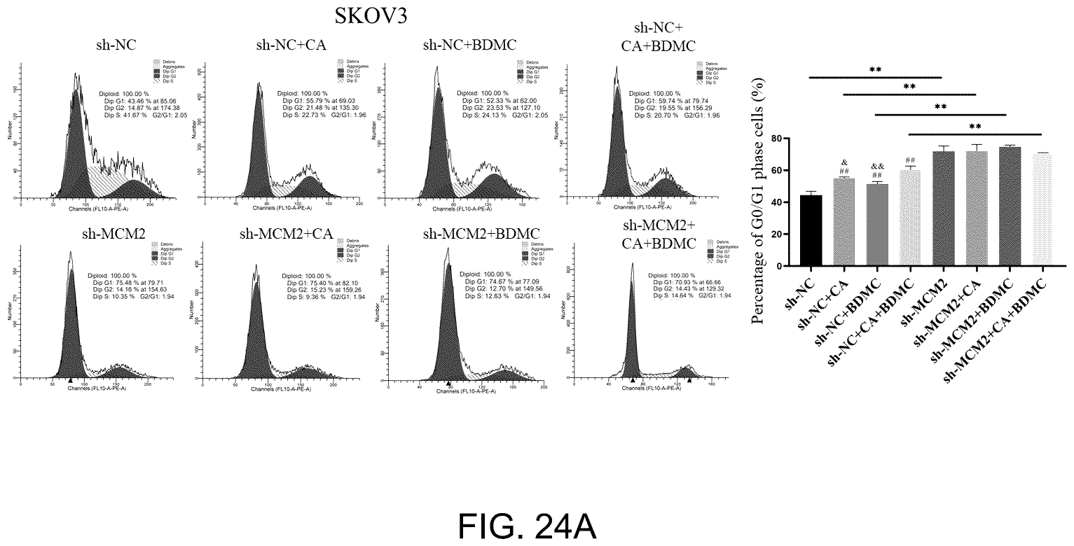

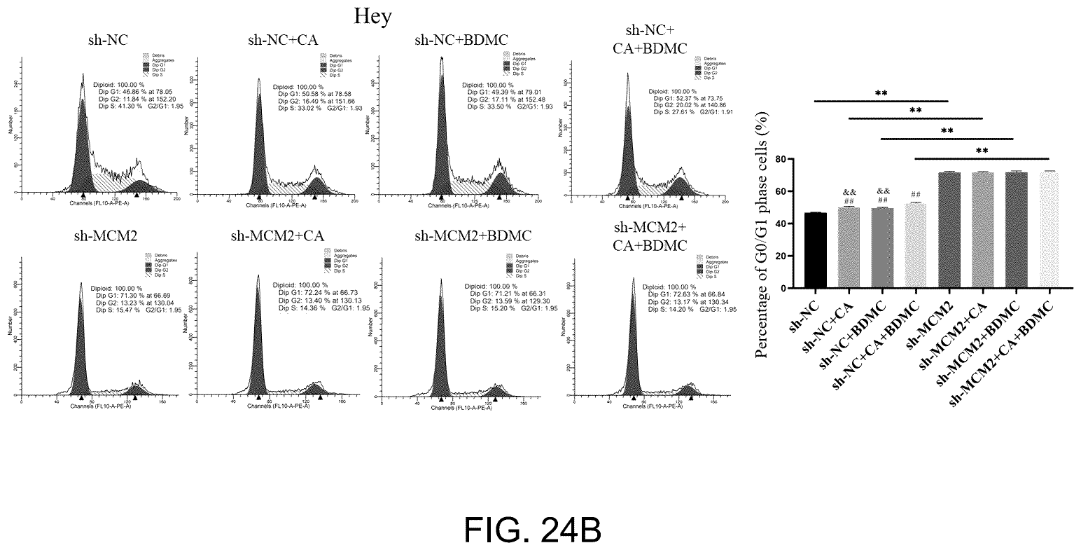

The cell cycle changes of CA, BDMC, and combined administration intervention on cell migration after knocking down MCM2 in SKOV3 and Hey ovarian cancer cells was shown in FIGS. 24A-24B. A represented the cell cycle of SKOV3 cells; and B represented the Hey cell cycle. The lentivirus empty vector infected sh-NC cells of SKOV3 and Hey ovarian cancer cells resulted in cell cycle arrest in G0/G1 phase after intervention with CA, BDMC, and combined administration. The percentage of G0/G1 phase cells in the sh-NC+CA group, sh-NC+BDMC group, and sh-NC+CA+BDMC group of the two types of cells significantly increased compared to the sh-NC group (P<0.01). The percentage of G0/G1 phase cells in the sh-NC+CA+BDMC group was significantly higher than that in the sh-NC+CA group and sh-NC+BDMC group after combined administration (P<0.05, P<0.01). After knocking down MCM2, there was no significant change in the percentage of G0/G1 phase cells after intervention with CA, BDMC, and combined administration. However, there was no significant change in the percentage of G0/G1 phase cells between the sh-MCM2+CA group, sh-MCM2+BDMC group, and sh-MCM2+CA+BDMC group compared to the sh-MCM2 group. The percentage of G0/G1 phase cells in the sh-MCM2+CA+BDMC group showed no significant change compared to the sh-MCM2+CA group and sh-MCM2+BDMC group in the combined administration group. Meanwhile, under the same administration treatment conditions, the percentage of G0/G1 phase cells in the sh-MCM2 cell treatment group significantly increased compared to the sh-NC cell treatment group (P<0.01).

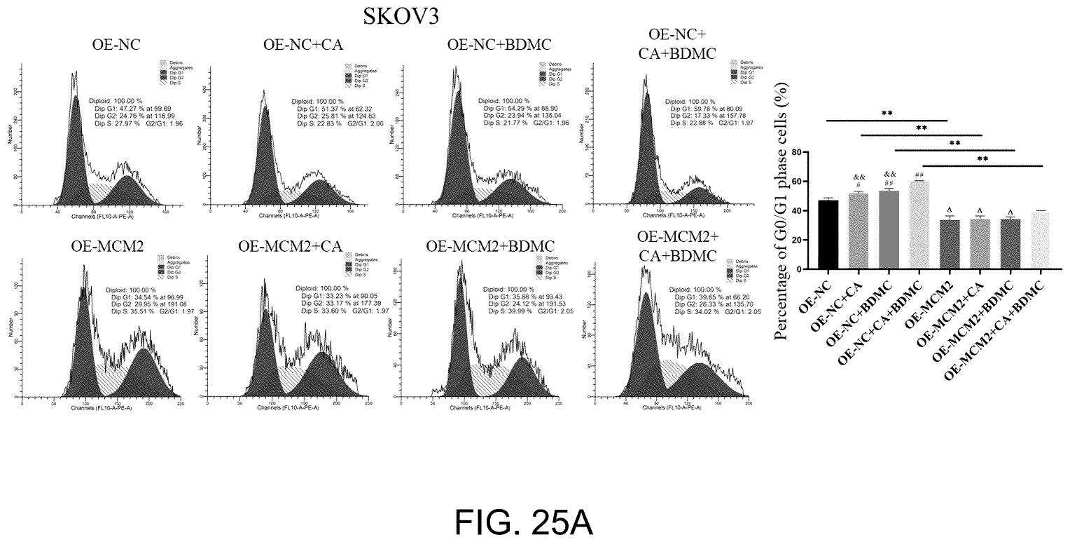

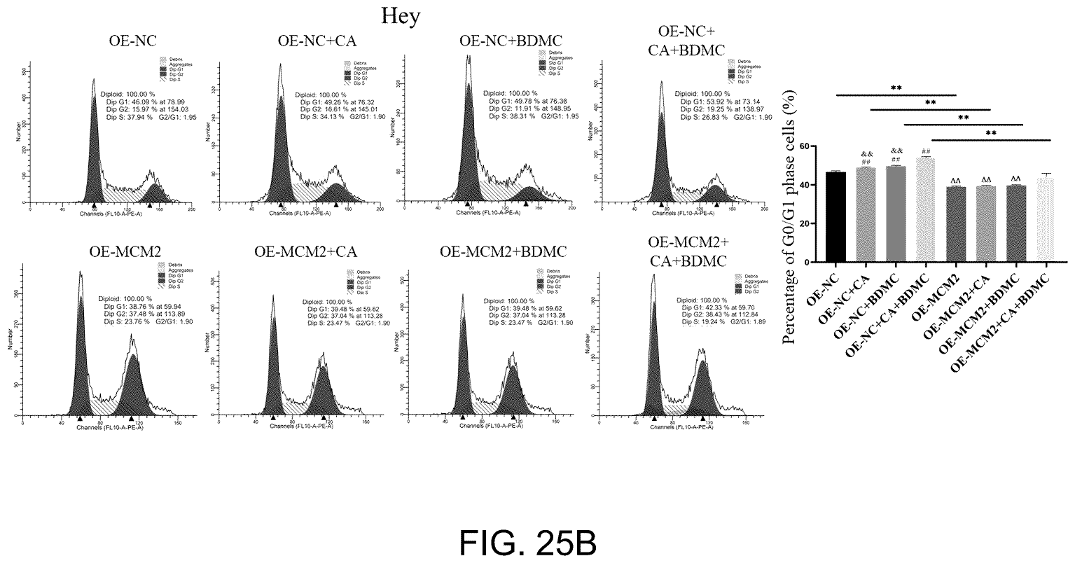

The cell cycle changes of SKOV3 and Hey ovarian cancer cells after overexpression of MCM2 and intervention with CA, BDMC, and combined administration were shown in FIGS. 25A-25B. A represented the cell cycle of SKOV3 cells; and B represented the Hey cell cycle. The lentivirus empty vector infected OE-NC cells of SKOV3 and Hey ovarian cancer cells resulted in cell cycle arrest in G0/G1 phase after intervention with CA, BDMC, and combined administration. The percentage of G0/G1 phase cells in the OE-NC+CA group, OE-NC+BDMC group, and OE-NC+CA+BDMC group of the two types of cells significantly increased compared to the OE-NC group (P<0.05, P<0.01). The percentage of G0/G1 phase cells in the OE-NC+CA+BDMC group was significantly increased compared to the OE-NC+CA group and OE-NC+BDMC group in the combined administration group (P<0.01). After overexpression of MCM2, there was no significant change in the percentage of G0/G1 phase cells in the OE-MCM2+CA group and OE-MCM2+BDMC group after single drug intervention with CA and BDMC. The percentage of G0/G1 phase cells in the OE-MCM2+CA group and OE-MCM2+BDMC group of the two types of cells showed no significant change compared to the OE-MCM2 group, while the percentage of G0/G1 phase cells in the OE-MCM2+CA+BDMC group after combined administration showed a significant increase compared to the OE-MCM2 group, OE-MCM2+CA group, and OE-MCM2+BDMC group (P<0.05, P<0.01). Meanwhile, under the same administration treatment conditions, the percentage of G0/G1 phase cells in OE-MCM2 cells significantly decreased compared to OE-NC cells (P<0.05, P<0.01). This experiment showed that CA, BDMC, and combined administration intervention could increase the percentage of G0/G1 phase cells in the ovarian cancer cells, block cells in G0/G1 phase, and the combined administration group was more effective than each single drug group; and after knocking down and overexpressing MCM2, the drug efficacy was weakened, the percentage of G0/G1 phase cells in each group intervened after knocking down MCM2 showed no significant change, while only the combined administration group showed efficacy after overexpression of MCM2.

### 2. CA, BDMC, and Combined Administration Affected the Cell Cycle and Expression of EMT Related Proteins in Ovarian Cancer Cells Through MCM2

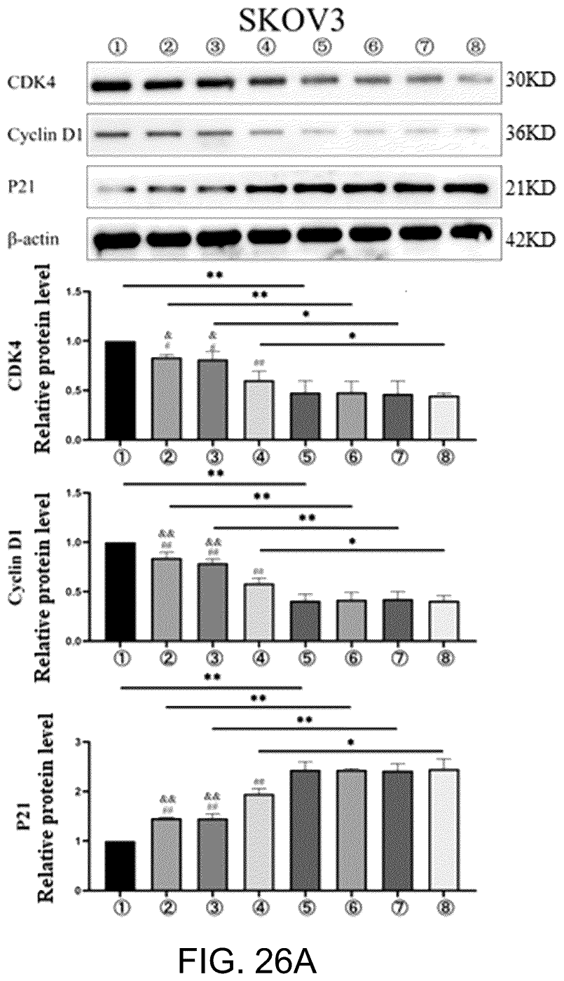

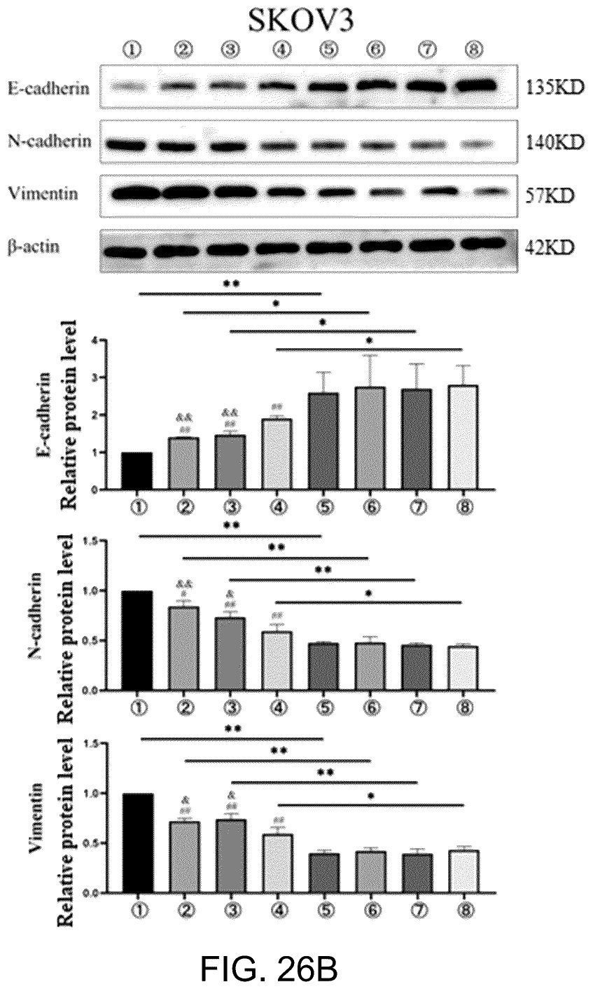

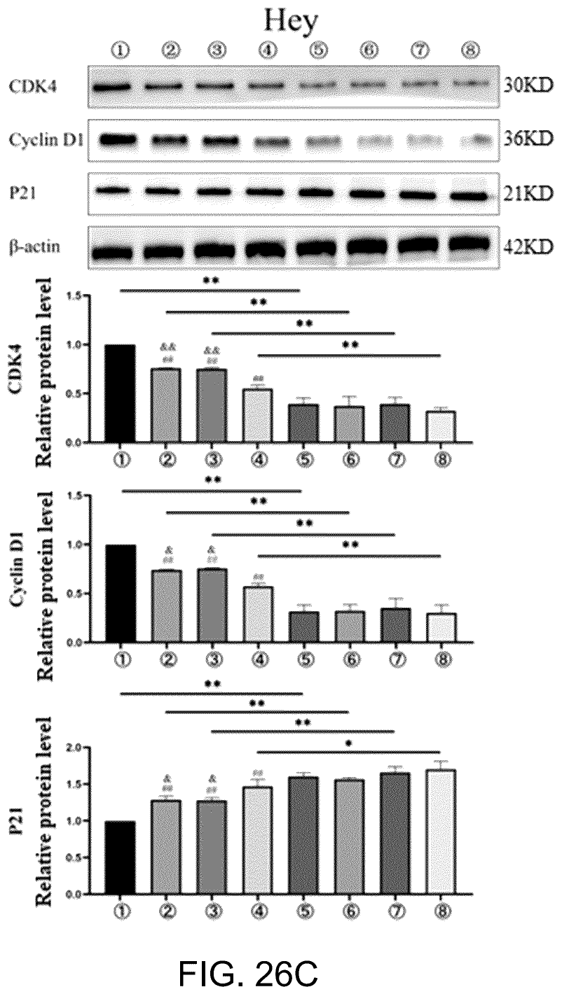

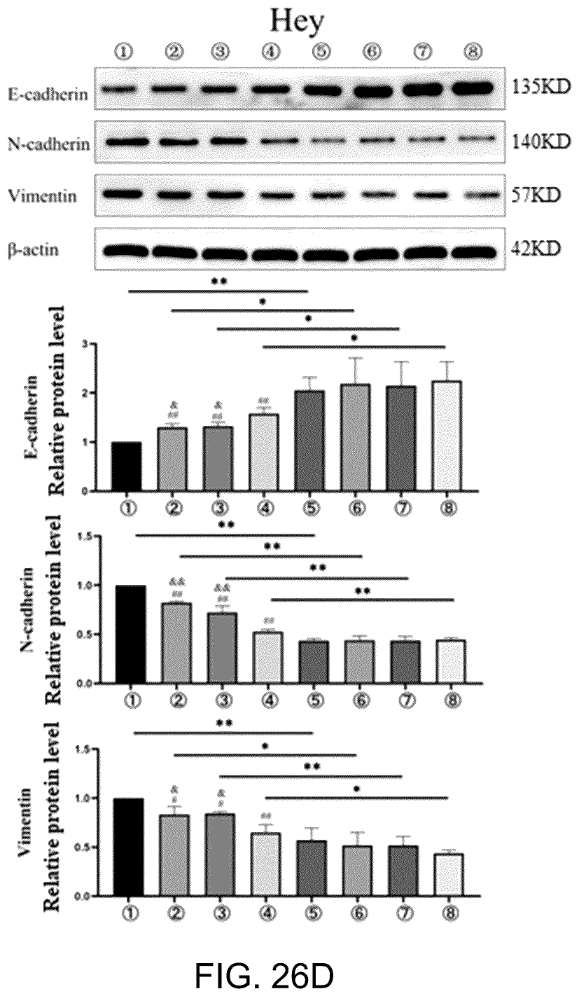

The impact of CA, BDMC, and combined administration intervention on cell cycle and EMT related proteins after knocking down MCM2 in SKOV3 and Hey ovarian cancer cells was shown in FIGS. 26A-26D. The expression of cell cycle related proteins (FIGS. 26A, 26C): the expression of CDK4 and Cyclin D1 in the sh-NC+CA group, sh-NC+BDMC group, and sh-NC+CA+BDMC group of SKOV3 and Hey ovarian cancer cells was significantly decreased compared to the sh-NC group (P<0.05, P<0.01). The expression of CDK4 and Cyclin D1 in the sh-NC+CA+BDMC group was significantly decreased compared to the sh-NC+CA group and the sh-NC+BDMC group in the combined administration group (P<0.05, P<0.01); and the expression of P21 in the sh-NC+CA group, sh-NC+BDMC group, and sh-NC+CA+BDMC group was significantly increased compared to the sh-NC group (P<0.01). The expression of P21 in the sh-NC+CA+BDMC group was significantly increased compared to the sh-NC+CA group and the sh-NC+BDMC group in the combined administration group (P<0.05, P<0.01). After knocking down MCM2, there was no significant change in the expression of CDK4, Cyclin D1, and P21 in cells after intervention with CA, BDMC, and combined administration. However, there was no significant change in the expression of CDK4, Cyclin D1, and P21 in the sh-MCM2+CA group, sh-MCM2+BDMC group, and sh-MCM2+CA+BDMC group compared to the sh-MCM2 group. The expression of CDK4, Cyclin D1, and P21 in the sh-MCM2+CA+BDMC group showed no significant changes compared to the sh-MCM2+CA group and sh-MCM2+BDMC group in the combined administration group. Meanwhile, under the same administration treatment conditions, the expression of CDK4 and Cyclin D1 in the sh-MCM2 cell treatment group was significantly decreased compared to the sh-NC cell treatment group (P<0.05, P<0.01); and the expression of P21 in the sh-MCM2 cell treatment group was significantly increased compared to the sh-NC cell treatment group (P<0.05, P<0.01).

The expression of EMT related proteins (FIGS. 26B, 26D): the E-cadherin expression in the sh-NC+CA group, sh-NC+BDMC group, and sh-NC+CA+BDMC group of SKOV3 and Hey ovarian cancer cells was significantly increased compared to the sh-NC group (P<0.01). The expression of E-cadherin in the sh-NC+CA+BDMC group was significantly increased compared to the sh-NC+CA group and the sh-NC+BDMC group in the combined administration group (P<0.05, P<0.01); and the expression of N-cadherin and Vimentin in the sh-NC+CA group, sh-NC+BDMC group, and sh-NC+CA+BDMC group was significantly decreased compared to the sh-NC group (P<0.05, P<0.01). The expression of N-cadherin and Vimentin in the sh-NC+CA+BDMC group was significantly decreased compared to the sh-NC+CA group and sh-NC+BDMC group in the combined administration group (P<0.05, P<0.01). After knocking down MCM2, there was no significant change in the expression of E-cadherin, N-cadherin, and Vimentin in cells after intervention with CA, BDMC, and combined administration. The expression of E-cadherin, N-cadherin, and Vimentin in the sh-MCM2+CA group, sh-MCM2+BDMC group, and sh-MCM2+CA+BDMC group of the two types of cells showed no significant change compared to the sh-MCM2 group. The expression of E-cadherin, N-cadherin, and Vimentin in the sh-MCM2+CA+BDMC group showed no significant changes compared to the sh-MCM2+CA group and sh-MCM2+BDMC group in the combined administration group. Meanwhile, under the same administration treatment conditions, the expression of E-cadherin in the sh-MCM2 cell treatment group was significantly increased compared to the sh-NC cell treatment group (P<0.05, P<0.01); and the expression of N-cadherin and Vimentin in the sh-MCM2 cell treatment group was significantly decreased compared to the sh-NC cell treatment group (P<0.05, P<0.01).

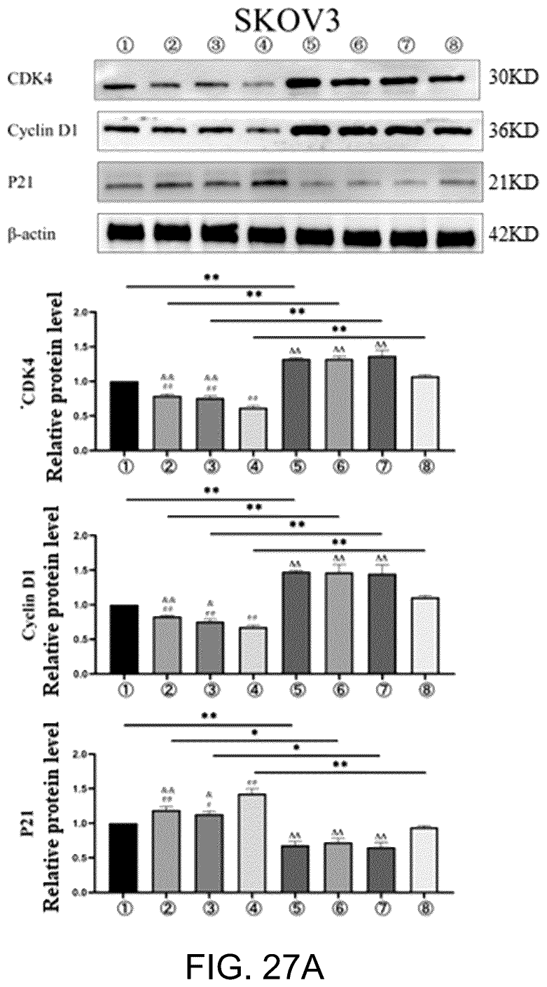

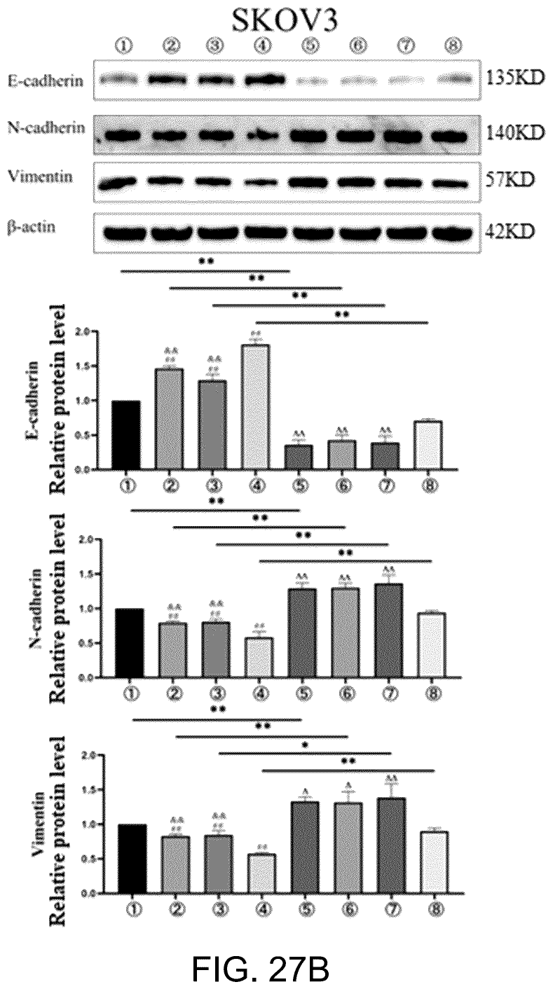

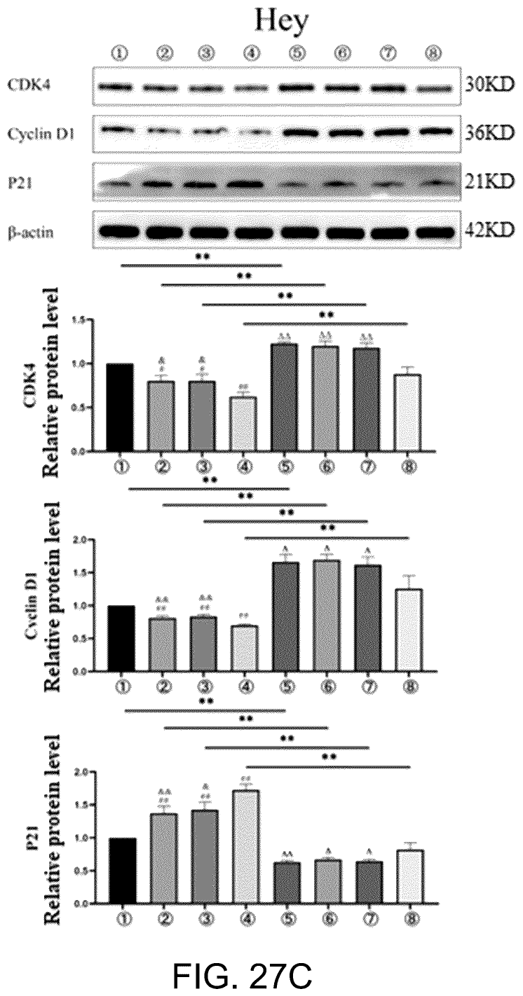

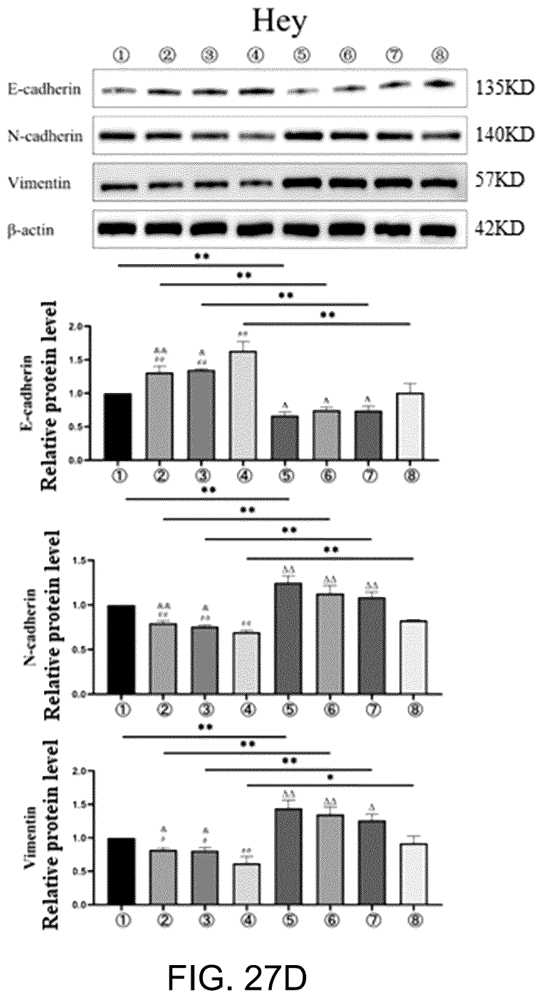

The impact of CA, BDMC, and combined administration intervention on cell cycle and EMT related proteins after overexpression of MCM2 in SKOV3 and Hey ovarian cancer cells were shown in FIGS. 27A-27D. The expression of cell cycle related proteins (FIGS. 27A, 27C): the expression of CDK4 and Cyclin D1 in the OE-NC+CA group, OE-NC+BDMC group, and OE-NC+CA+BDMC group of SKOV3 and Hey ovarian cancer cells was significantly decreased compared to the OE-NC group (P<0.05, P<0.01). The expression of CDK4 and Cyclin D1 in the OE-NC+CA+BDMC group was significantly decreased compared to the OE-NC+CA group and OE-NC+BDMC group in the combined administration group (P<0.05, P<0.01); and the expression of P21 in the OE-NC+CA group, OE-NC+BDMC group, and OE-NC+CA+BDMC group was significantly increased compared to the OE-NC group (P<0.05, P<0.01). The expression of P21 in the OE-NC+CA+BDMC group was significantly increased compared to the OE-NC+CA group and OE-NC+BDMC group in the combined administration group (P<0.05, P<0.01). After overexpression of MCM2, there was no significant change in the expression of CDK4, Cyclin D1, and P21 in cells after single drug intervention with CA and BDMC. The expression of CDK4, Cyclin D1, and P21 in the OE-MCM2+CA group and OE-MCM2+BDMC group of the two types of cells showed no significant change compared to the OE-MCM2 group. The expression of CDK4 and Cyclin D1 in the OE-MCM2+CA+BDMC group was significantly decreased compared to the OE-MCM2 group, OE-MCM2+CA group, and OE-MCM2+BDMC group in the combined administration group (P<0.05, P<0.01); and the expression of P21 in the OE-MCM2+CA+BDMC group was significantly increased compared to the OE-MCM2 group, OE-MCM2+CA group, and OE-MCM2+BDMC group (P<0.05, P<0.01). Meanwhile, under the same administration treatment conditions, the expression of CDK4 and Cyclin D1 in the OE-MCM2 cell treatment group was significantly increased compared to the OE-NC cell treatment group (P<0.01); and the expression of P21 in the OE-MCM2 cell treatment group was significantly decreased compared to the OE-NC cell treatment group (P<0.05, P<0.01).

The expression of EMT related proteins (FIGS. 27B, 27D): the E-cadherin expression in the OE-NC+CA group, OE-NC+BDMC group, and OE-NC+CA+BDMC group of SKOV3 and Hey ovarian cancer cells was significantly increased compared to the OE-NC group (P<0.01). The expression of E-cadherin in the OE-NC+CA+BDMC group was significantly increased compared to the OE-NC+CA group and OE-NC+BDMC group in the combined administration group (P<0.05, P<0.01); and the expression of N-cadherin and Vimentin in the OE-NC+CA group, OE-NC+BDMC group, and OE-NC+CA+BDMC group was significantly decreased compared to the OE-NC group (P<0.05, P<0.01). The expression of N-cadherin and Vimentin in the OE-NC+CA+BDMC group was significantly decreased compared to the OE-NC+CA group and OE-NC+BDMC group in the combined administration group (P<0.05, P<0.01). After overexpression of MCM2, there was no significant change in the expression of CDK4, Cyclin D1, and P21 in cells after single drug intervention with CA and BDMC. The expression of E-cadherin, N-cadherin, and Vimentin in the OE-MCM2+CA group and OE-MCM2+BDMC group showed no significant change compared to the OE-MCM2 group. The expression of E-cadherin in the OE-MCM2+CA+BDMC group was significantly increased compared to the OE-MCM2 group, OE-MCM2+CA group, and OE-MCM2+BDMC group in the combined administration group (P<0.05, P<0.01); and the expression of N-cadherin and Vimentin in the OE-MCM2+CA+BDMC group was significantly decreased compared to the OE-MCM2 group, OE-MCM2+CA group, and OE-MCM2+BDMC group (P<0.05, P<0.01). Meanwhile, under the same administration treatment conditions, the expression of E-cadherin in the OE-MCM2 cell treatment group was significantly decreased compared to the OE-NC cell treatment group (P<0.01); and the expression of N-cadherin and Vimentin in the OE-MCM2 cell treatment group was significantly increased compared to the OE-NC cell treatment group (P<0.05, P<0.01). This experiment showed that CA, BDMC, and combined administration intervention could affect the cell cycle and expression of EMT related proteins in the ovarian cancer cells, and the combined administration group is more effective than each single drug group; and after knocking down and overexpression of MCM2, the drug efficacy was weakened. After knocking down MCM2, there was no significant change in each group after intervention with administration. After overexpression of MCM2, only the combined administration group showed drug efficacy.

### 3. Immunohistochemical Assay for Detecting Tumor Body Cell Cycle Related Proteins in Tumor Bearing Nude Mice

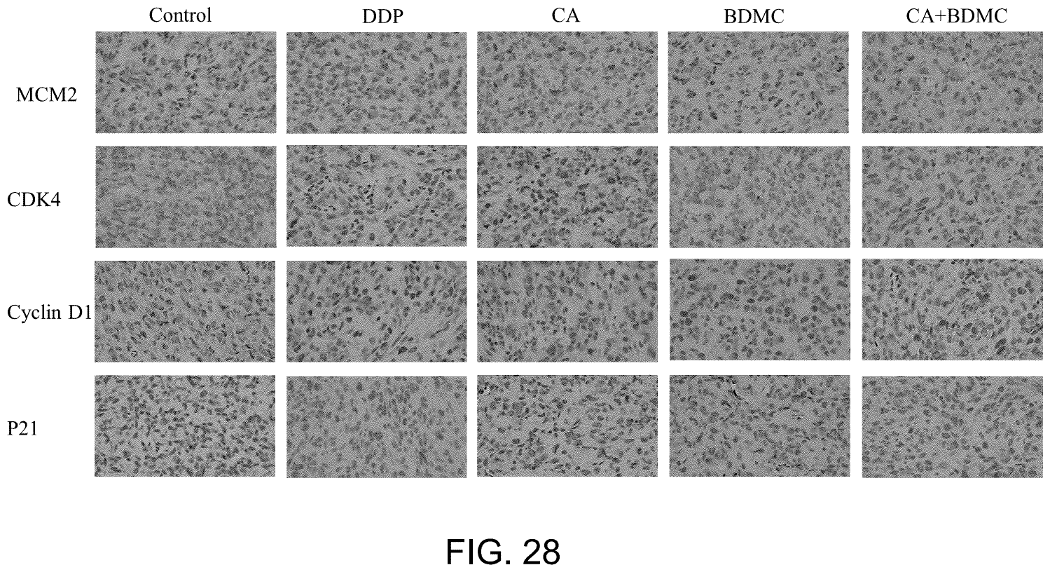

The immunohistochemistry of tumor body cyclin in tumor bearing nude mice after intervention with DDP, CA, BDMC, and combined administration was shown in FIG. 28. The image was magnified by 400×, with a scale of 10 μM. The expression of MCM2, CDK4, and Cyclin D1 proteins in the CA group, BDMC group, and CA+BDMC group decreased compared to the Control group; and the expression of MCM2, CDK4, and Cyclin D1 proteins in the CA+BDMC group decreased compared to the CA group and BDMC group. The expression of P21 protein in the CA group, BDMC group, and CA+BDMC group increased compared to the Control group; and the expression of P21 protein in the CA+BDMC group increased compared to the CA group and BDMC group.

### 4. Western Blot Assay for Detecting Tumor Body Cyclin and EMT Related Proteins in Tumor Bearing Nude Mice

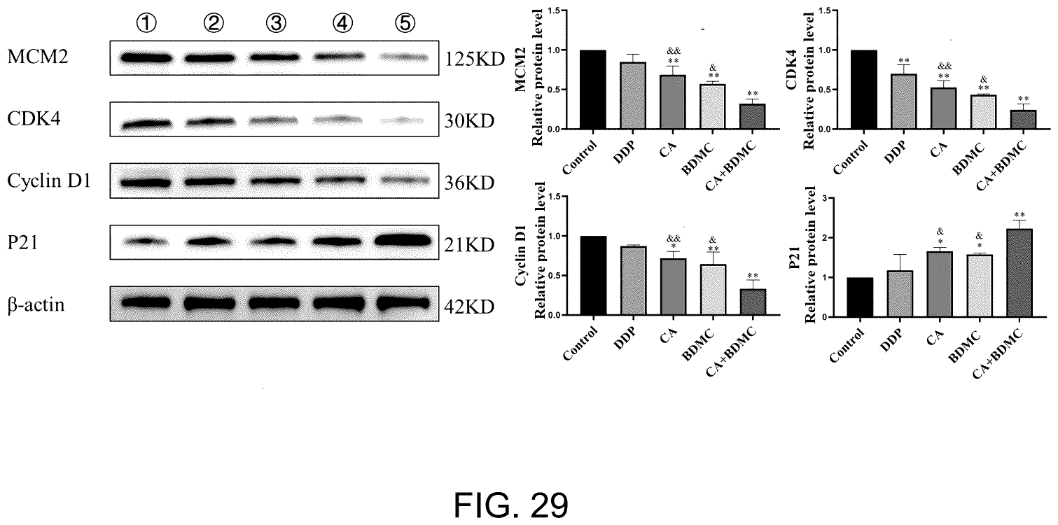

The expression of nude mice tumor body cyclin and EMT related proteins in tumor bearing nude mice after intervention with DDP, CA, BDMC, and combined administration was shown in FIG. 29. In the figure, 1 represented the Control group; 2 represented the DDP group; 3 represented the CA group; 4 represented the BDMC group; and 5 represented the CA+BDMC group.

The expression of cell cycle related proteins: the expression of MCM2, CDK4, and Cyclin D1 proteins in the CA group, BDMC group, and CA+BDMC group was significantly decreased compared to the Control group (P<0.05, P<0.01); the expression of CDK4 protein in the DDP group was significantly decreased compared to the Control group (P<0.01); and the expression of MCM2, CDK4, and Cyclin D1 proteins in the CA+BDMC group was significantly decreased compared to the CA group and BDMC group (P<0.05, P<0.01). The expression of P21 protein in the CA group, BDMC group, and CA+BDMC group was significantly increased compared to the Control group (P<0.05, P<0.01); and the expression of P21 protein in the CA+BDMC group was significantly increased compared to the CA group and BDMC group (P<0.05).

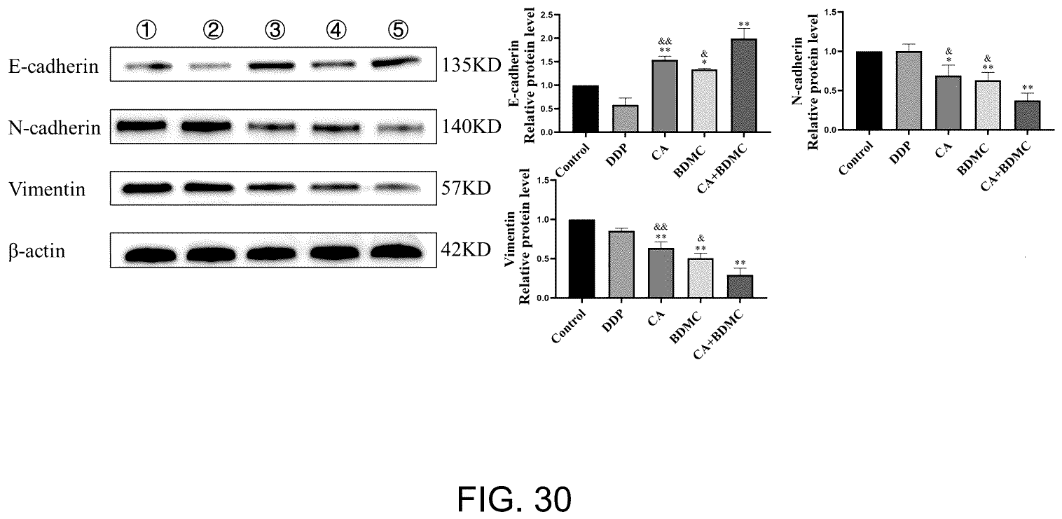

The expression of EMT related proteins was shown in FIG. 30: \textcircled{1} represented the Control group; \textcircled{2} represented the DDP group; \textcircled{3} represented the CA group; \textcircled{4} represented the BDMC group; and \textcircled{5} represented the CA+BDMC group.

The expression of E-cadherin protein in the CA group, BDMC group, and CA+BDMC group was significantly increased compared to the Control group (P<0.05, P<0.01); and the expression of E-cadherin protein in the CA+BDMC group was significantly increased compared to the CA group and BDMC group (P<0.05, P<0.01). The expression of N-cadherin and Vimentin proteins in the CA group, BDMC group, and CA+BDMC group was significantly decreased compared to the Control group (P<0.05, P<0.01); and the expression of N-cadherin and Vimentin proteins in the CA+BDMC group was significantly decreased compared to the CA group and BDMC group (P<0.05, P<0.01). This experiment showed that CA, BDMC, and combined administration could affect the cell cycle and expression of EMT related proteins in tumor bearing nude mice, and the combined administration was more effective than single drug.

The above-mentioned embodiments only express the specific embodiments of the present application, and their descriptions are more specific and detailed, but should not be understood as limiting the scope of protection of the present application. It should be pointed out that for those skilled in the art, several modifications and improvements can be made without departing from the technical concept of the present application, which are within the scope of protection of the present application.

What is claimed is:

1. An anti-ovarian cancer pharmaceutical composition comprising a Curcuma zedoaria extract.

2. The anti-ovarian cancer pharmaceutical composition according to claim 1, further comprising an Astragalus membranaceus extract.

3. The anti-ovarian cancer pharmaceutical composition according to claim 2, wherein the Astragalus membranaceus extract comprises calycosin, and the Curcuma zedoaria extract comprises bisdemethoxycurcumin.

4. The anti-ovarian cancer pharmaceutical composition according to claim 3, wherein a ratio of the calycosin to the bisdemethoxycurcumin is 16:1.

5. The anti-ovarian cancer pharmaceutical composition according to claim 3, wherein the anti-ovarian cancer pharmaceutical composition targets an MCM2 gene.

6. The anti-ovarian cancer pharmaceutical composition according to claim 5, wherein the anti-ovarian cancer pharmaceutical composition targets the MCM2 gene to block cell cycle regulation and inhibit an ovarian cancer cell by affecting an EMT pathway.

7. A use of an MCM2 gene as an action target of an anti-ovarian cancer pharmaceutical composition in development and screening of a drug for preventing or treating the ovarian cancer, wherein the anti-ovarian cancer pharmaceutical composition targets the MCM2 gene to block cell cycle regulation and inhibit an ovarian cancer cell by affecting an EMT pathway.

8. The use according to claim 7, wherein the anti-ovarian cancer pharmaceutical composition affects levels of CDK4, Cyclin D1, and P21, causing formation of a Cyclin D1-CDK4/6 complex and blocking the cell cycle.

9. The use according to claim 7, wherein the anti-ovarian cancer pharmaceutical composition affects levels of E-cadherin, N-cadherin, and Vimentin to regulate the EMT pathway, reducing proliferation, migration, and invasion abilities of the ovarian cancer cells.

10. The anti-ovarian cancer pharmaceutical composition according to claim 4, wherein the anti-ovarian cancer pharmaceutical composition targets an MCM2 gene.

11. The anti-ovarian cancer pharmaceutical composition according to claim 10, wherein the anti-ovarian cancer pharmaceutical composition targets the MCM2 gene to block cell cycle regulation and inhibit an ovarian cancer cell by affecting an EMT pathway.
